(19) United States
(12) Patent Application Publication
(10) Pub. No.: US 2017/0044269 A1
(43) Pub. Date: Feb. 16, 2017

(54) ADENOVIRAL TARGETING, COMPOSITIONS AND METHODS THEREFOR

(71) Applicant: Washington University, Saint Louis, MO (US)

(72) Inventors: David Curiel, Saint Louis, MO (US); Sergey Kaliberov, Saint Louis, MO (US)

(21) Appl. No.: 15/296,522

(22) Filed: Oct. 18, 2016

Related U.S. Application Data
(63) Continuation of application No. PCT/US15/26627, filed on Apr. 20, 2015.
(60) Provisional application No. 61/981,462, filed on Apr. 18, 2014.

(51) Int. Cl. C07K 16/30; C12N 15/86; C07K 14/005

(52) U.S. Cl. CPC C07K 16/3007; C07K 14/005; C12N 15/86; C07K 2317/22; C07K 2317/622; C07K 2319/00; C12N 2710/10322; C12N 2710/10332; C12N 2710/10345; C12N 2810/859; C12N 2830/008; C12N 2830/85; C07K 2319/73; C07K 2319/33

(57) ABSTRACT

Polypeptides are disclosed comprising, in N-terminal-to-C-terminal order: an N-terminal segment of Ad5 fiber tail sequence; at least 2 pseudorepeats of an Ad5 fiber shaft domain sequence; a portion of a third Ad5 fiber shaft domain sequence; a carboxy-terminal segment of a 14 fibritin bacteriophage trimerization domain sequence; a linker sequence; and a camelid single chain antibody sequence. A camelid single chain antibody sequence can be against a human carcinoembryonic antigen. Also disclosed are nucleic acids encoding these polypeptides, and adenovirus vectors comprising the polypeptides. Methods are disclosed for treating a neoplastic disease. These methods can comprise administering an adenovirus vector comprising a disclosed polypeptide. Also disclosed are methods of targeting a vector to CEA-expressing cells. These methods comprise administering an adenovirus vector comprising a disclosed polypeptide. Methods can further comprise subjecting a subject to ionizing radiation in an amount effective for inducing CEA overexpression.

### CROSS REFERENCE TO PRIOR APPLICATIONS

This application claims benefit of and priority to PCT/US15/26627, filed Apr. 20, 2015, and to U.S. Provisional Application 61/981,462, filed Apr. 18, 2014, each of which is hereby incorporated by reference in its entirety.

### STATEMENT REGARDING FEDERALLY SPONSORED RESEARCH OR DEVELOPMENT

This invention was made with government support under AIl01403 awarded by the National Institutes of Health. The government has certain rights in the invention.

### INCORPORATION BY REFERENCE OF SEQUENCE LISTING

The Sequence Listing, which is a part of the present disclosure, includes a computer readable form and a written sequence listing comprising nucleotide and/or amino acid sequences. The subject matter of the Sequence Listing is incorporated herein by reference in its entirety. The information recorded in electronic form furnished under Rule 13ter is identical to the sequence listing as contained in the international application.

### Introduction

Human Ad serotype 5 (Ad5), which is associated with relatively mild diseases, can infect a wide range of cell types with low oncogenic potential. There are also methods for generation of Ad5 recombinant viruses for tumor-specific gene delivery (Harriett, B. G., et al., Biochimica et Biophysica Acta, 1575, 1-14, 2002). Human clinical trials have validated the overall safety of Ad5-based cancer gene therapy and have demonstrated evidence of clinical efficacy (Vasey. P. A., et al., Journal of Clinical Oncology, 2002, 20,1562-1569 and Kirn, D., et al., Nature Medicine, 4, 1341-1342, 1998). Ad5 tropism is dictated by recognition of the native primary receptor “coxsackie-and-adenovirus receptor” (CAR) via the knob domain of the capsid protein fiber (Henning, P., et al., J. Gen. Virol, 87, 3151-3160, 2006). Ad5-based gene therapy has been limited due to low CAR expression in tumor cells.

Ad5 tropism has been modified using both molecular adapter proteins and genetic capsid modifications (Glasgow, J. N., et al., Cancer Gene Ther., 13, 830-844, 2006, and Noureddini, S. C., et al., Mol. Pharm., 1998, 2, 341-347, 1998). CAR-independent Ad5 transduction with enhanced, vector infectivity of tumor cells has been demonstrated (Dmitriev J., et al. 1998 J. Virol., 72, 9706-9713, 1998). Some targeting moieties have been employed for rendering recombinant Ad vectors tumor-selective (reviewed in Beatty, M. S., et al., Advances in Cancer Research, 115, 39-67, 2012).

A molecular adaptor retargeting approach in conjunction with anti-tumor single-chain Fvs has been used with tumor-selective gene delivery (Kashentseva, E. A., et al. Cancer Res. 62, 609-616, 2002; Barker, S. D., et al. Gene Ther. 10, 1198-1204, 2003; Li, H. J., et al. Cancer Res 67; 5354-5361, 2007; and Li, H. J., et al. Cancer Res. 69, 554-564, 2009). Genetic modifications of Ad5 capsid have been used to incorporate anti-tumor scFv into Ad particles via a “fiber replacement” approach (Belousova, N., et al., J. Virol. 77, 11367-11377, 2003). However, whereas this strategy provides a means to incorporate large targeting ligands into the Ad capsid, a loss of binding specificity was observed. Additionally, the available repertoire of anti-tumor specificities of seFvs is limited which restricts this approach.

Immunoglobulins (Ig) derived from the camelid family have heavy-chains as the basis of antigen (Ag) recognition and binding (“nanobodies,” Hamers-Casterman, C., et al., Nature 363,446-448, 1993; Vaneycken, I., et al., J. Nucl. Med 51, 110-1106, 2010; Revets, H., et al., Expert Opin. Bio. Ther. 5, 111-124, 2005), Some researchers have developed non-immune single domain antibody (sdAb) libraries and have employed them for biopanning (Shao, C. Y., et al. Mol. Immunol. 44, 656-665, 2007; Wei, G., et al., PLoS One 6, e28309, 2011; Verheesen, P., et al., Biochim. Biophys. Acta 1764, 1307-1319, 2006; Goldman, E. R., et al., Anal Chem. 78, 8245-8255, 2006; Reiter, Y., et al., J. Mol. Biol. 290, 685-698, 1999). Some engineered sdAb fusion proteins have demonstrated tumor targeting in model systems (Cortez-Retamozo, V., et al., Cancer Res. 64, 2853-2857, 2004 and Cortex-Retamozo, V., et al. Int'l. J. Cancer 98, 456-462, 2002).

PCT Application PCT/US2013/031002 (WO2013138505 A1) of O'Shea, C., et al. discloses adenoviral cancer cell-targeting constructs comprising a CEA-VHH operably linked to FKBP. This PCT application does not disclose a fiber including both a fibritin domain and a single chain antibody domain.

Krasnykh, V., et al., J. Virol. 75, 4176-4183, 2001 discloses a human Ad5 incorporating chimeric fiber-fibritin proteins to target artificial receptor molecules. Noureddini, S. C. and Curiel, D. T., Mol. Pharm. 2, 341-347, 2005 reviews genetic targeting strategies for Ad5. U.S. Pat. No. 6,210,946 to Curiel, D. T., et al. discloses an adenovirus including a chimeric fiber. U.S. Pat. No. 6,824,771 to Curiel, D. T., et al. discloses an adenovirus including a fiber substitute protein. None of Krasnykh, V., et al., Virol. 75, 4176-4183, 2001, Noureddini, S. C., et al., U.S. Pat. No. 6,210,946 or U.S. Pat. No. 6,824,771 discloses incorporation of camelid single chain antibodies into an adenovirus vector.

Noureddini, S. C., et al., Virus Res. 116, 185-195, 2006 Epub 2005 Nov 15 (abstract) discloses an Ad5-based vector but does not disclose camelid single chain antibodies.

U.S. Pat. No. 6,555,368 to Curiel, D. T., et al discloses recombinant adenoviral vectors in which a single-chain antibody has been introduced into the minor capsid proteins, pIIIa or pIX, to target the adenoviral vector to a particular cell type. This patent does not disclose use of camelid single chain antibodies.

Poulin, K. L., et al., J. Virol., 84, 10074-10086, 2010 discloses modification of pIX capsid proteins, but does not disclose modification of the fiber protein.

Matsui, H., et al. Biomaterials, 34, 4191-4201, 2013 discloses modifications in Ad capsid proteins using sdAb mimic-monobodies based on the 10th fibronectin type III domain.

Pereboeva, L., et al., Gene Ther., 14, 627-637, 2007 discloses modification of adenoviral vectors to target EGFR-expressing cells in vitro and does not disclose camelid antibodies.

Revets, H., el al. Expert Opin. Biol. Ther., 5, 111-124, 2005 discloses single chain antibodies but not fiber-fibritin chimeric proteins with camelid antibodies as targeting ligands.

Radiation can be directed at a specific site using external or internal sources as a method of treating cancer cells. However, this approach can be limited by systemic toxicity and radiosensitization of normal tissues. There is a need for identification of targeted therapy agents that could enhance the efficacy of radiation treatment for multimodality therapies.

There is also a need for successful re-targeting of gene transfer vectors to achieve the gene therapy pharmacologic mandates of efficient and specific target cell transduction. Biologic issues have confounded the logical and direct exploitation of antibody species to retarget Ad vectors. There is a need for Ad-targeting technology to facilitate the application of cancer gene therapies to the clinical context of metastatic disease (Khare, R., et al., Current Gene Therapy, 11, 241-258, 2011).

### SUMMARY

The present inventors disclose modified Ad5 vectors with altered tropisms. In various embodiments, an Ad knob sequence can be replaced with camelid antibody species to alter tropism for cell-specific targeted gene transfer.

In some embodiments, the present teachings include a genetically-modified adenoviral vector comprising a chimeric polypeptide comprising a de-knobbed Ad5 fiber, a T4 bacteriophage fibritin trimerization sequence and a camelid (VHH single chain) antibody sequence. An adenoviral vector of the present teachings can be used in conjunction with many different camelid antibodies. For example and without limitation, in various configurations, a camelid antibody of the present teachings can be directed against a cell-surface antigen, such as human carcinoembryonic antigen (“hCEA,” a human tumor antigen), or Nb-DC1.8 which can recognize bone marrow-derived dendritic cells (De Groeve, K., et al., J. Nucl. Med., 51, 782-789, 2010). A vector of the present teachings can thus be used to effect targeted infection of a cell with an adenovirus, which can include a heterologous nucleic acid sequence and/or a polypeptide for delivery into a specific cell type.

In some embodiments, the present teachings include combinations of Ad and antibody species to accomplish specific gene transfer for gene therapy applications or for vaccines.

In some embodiments, an adenoviral vector of the present teachings can comprise an anti-hCEA VHH (such as VHH 122) in a de-knobbed Ad5 fiber-fibritin chimera. In some embodiments, introduction of a VHB such as an hCEA VHH and removal of the knob can provide an Ad5 vector which is targeted to tumor cells without the ability to bind CAR that may be present in non-tumor cells.

In some embodiments, the present teachings include adenovirus vectors comprising anti-human carcinoembryonic antigen (hCEA) single variable domains derived from a heavy chain (VHH) camelid antibody for targeted gene transfer.

In some embodiments, the present teachings include adenovirus vectors comprising a camelid antibody against a human dendritic cell marker, such as, without limitation Nb-DC1.8.

In some embodiments, the present teachings include immunization of a mammal to effect higher expression levels of a cell protein such as an interferon. In some configurations, splenocytes from mice immunized with a vector of the present teachings can exhibit statistically significant increases of INFγ expression relative to controls.

In some embodiments, adenoviral vectors of the present teachings can be targeted to dendritic cells.

In some embodiments, the present teachings include a panel of recombinant Ad5-based vectors expressing a fiber-fibritin-VHH fusion protein.

In some embodiments, the present teachings include methods and compositions for directing site-specific Ad-mediated therapeutic gene expression to a tumor by use of radiation to enhance the bioavailability of an anti-cancer gene therapy, In some configurations, these methods can reduce or minimize systemic toxicities.

In some embodiments, the present teachings include a polypeptide comprising, consisting essentially of, or consisting of, in N-terminal-to-C-terminal order: an N-terminal segment of Ad5 fiber tail sequence; at least 2 pseudorepeats of an Ad5 fiber shaft domain sequence; a portion of a third Ad5 fiber shaft domain sequence; a carboxy-terminal segment of a T4 fibritin bacteriophage trimerization domain sequence; a linker sequence; and a camelid single chain antibody sequence. In various configurations, a carboxy-terminal segment of a T4 fibritin bacteriophage trimerization domain sequence of a polypeptide of the present teachings can comprise an α-helical domain and a foldon domain.

In various configurations, an N-terminal segment of Ad5 fiber tail sequence of a polypeptide of the present teachings can be set forth as MKRARPSEDTFNPVYPYDTETGPPTVPFLTPPFVSPNGFQESPP (SEQ ID NO:1), a sequence having at least 70% sequence identity with SEQ ID NO:1 or about 70% sequence identity with SEQ ID NO:1 a sequence having at least 75% sequence identity with SEQ ID NO:1 or about 75% sequence identity with SEQ ID NO:1, a sequence having at least 80% sequence identity with SEQ ID NO:1 or about 80% sequence identity with SEQ ID NO:1, a sequence having at least 85% sequence identity with SEQ ID NO:1 or about 85% sequence identity with SEQ ID NO:1, a sequence having at least 90% sequence identity with SEQ ID NO:1 or about 90% sequence identity with SEQ ID NO:1, a sequence having at least 95% sequence identity with SEQ ID NO:1 or about 95% sequence identity with SEQ ID NO:1, a sequence having at least 96% sequence identity with SEQ ID NO:1, a sequence having at least 97% sequence identity with SEQ ID NO:1, a sequence having at least 98% sequence identity with SEQ ID NO:1, or a sequence having at least 99% sequence identity with SEQ ID NO:1.

In various configurations, at least 2 pseudorepeats of an Ad5 fiber shaft domain sequence of a polypeptide of the present teachings can be set forth as GVLSLRLSEPLVTSNGMALKMGNGLSLDEA (SEQ ID NO:2), a sequence having at least 70% sequence identity with SEQ ID NO:2 or about 70% sequence identity with SEQ ID NO:2, a sequence having at least 75% sequence identity with SEQ ID NO:2 or about 75% sequence identity with SEQ ID NO:2, a sequence having at least 80% sequence identity with SEQ ID NO.2 or about 80% sequence identity with SEQ ID NO:2, a sequence having at least 85% sequence identity with SEQ ID NO:2 or about 85% sequence identity with SEQ ID NO:2, a sequence having at least 90% sequence identity with SEQ ID NO:2 or about 90% sequence identity with SEQ ID NO:2, a sequence having at least 95% sequence identity with SEQ ID NO:2 or about 95% sequence identity with SEQ ID NO:2, a sequence having at least 96% sequence identity with SEQ ID NO:2, a sequence having at least 97% sequence identity with SEQ ID NO:2, a sequence having at least 98% sequence identity with SEQ ID NO:2, or a sequence having at least 99% sequence identity with SEQ ID NO:2.

In various configurations a portion of a third Ad5 fiber shaft domain sequence of a polypeptide of the present teachings comprise at least 8 contiguous amino acids of an Ad5 fiber shaft domain sequence, such as GNLTSQNV (SEQ ID NO:3), a sequence having at least 70% sequence identity with SEQ ID NO:3 or about 70% sequence identity with SEQ ID NO:3, a sequence having at least 75% sequence identity with SEQ ID NO:3 or about 75% sequence identity with SEQ ID NO:3, a sequence having at least 80% sequence identity with SEQ ID No:3 or about 80% sequence identity with SEQ ID NO:3, a sequence having at least 85% sequence identity with SEQ ID NO:3 or about 85% sequence identity with SEQ ID NO:3.

In various configurations, a carboxy-terminal segment of a T4 fibritin bacteriophage trimerization domain sequence of a polypeptide of the present teachings can be set forth as GYIPEAPRDGQAYVRKDGEWVLLSTFLSPA (SEQ ID NO:4), a sequence having at least 70% sequence identity with SEQ ID NO:4 or about 70% sequence identity with SEQ ID NO:4, a sequence having at least 75% sequence identity with SEQ ID NO:4 or about 75% sequence identity with SEQ ID NO:4, a sequence having at least 80% sequence identity with SEQ ID NO:4 or about 80% sequence identity with SEQ ID NO:4, a sequence having at least 85% sequence identity with SEQ ID NO:4 or about 85% sequence identity with SEQ ID NO:4, a sequence having at least 90% sequence identity with SEQ ID NO:4 or about 90% sequence identity with SEQ ID NO:4, a sequence having at least 95% sequence identity with SEQ ID NO:4 or about 95% sequence identity with SEQ ID NO:4, a sequence having at least 96% sequence identity with SEQ ID NO:4, a sequence having at least 97% sequence identity with SEQ ID NO:4, a sequence having at least 98% sequence identity with SEQ ID NO:4, or a sequence having at least 99% sequence identity with SEQ ID NO:4.

In various configurations, a linker sequence of a polypeptide of the present teachings can comprise the sequence (GlyₙSer)ₘ, wherein n is an integer from 2 to 6, and m is an integer from 1 to 5. In various configurations, a linker sequence of a polypeptide of the present teachings can be Gly-Gly-Gly-Gly-Ser (SEQ ID NO:5).

In various configurations, a camelid single chain antibody sequence of a polypeptide of the present teachings can be against a human carcinoembryonic antigen. In various configurations, a camelid single chain antibody sequence of a polypeptide of the present teachings can be selected from the group consisting of JJB-A3 set forth as QVQLVETGGGLVQPGGSLRLSCAASGRISDINAMGWYRQAPGKQRELVAAITSVGS NYVDSVKGRFTISKDNAKNTVYLQMYSLNPEDTAVYYCNTQCGTWLVCDGRDQW GKGTLVTVSSEPKTPKPQ (SEQ ID NO:6), a sequence having at least 70% sequence identity with SEQ ID NO:6 or about 70% sequence identity with SEQ ID NO:6, a sequence having at least 75% sequence identity with SEQ ID NO:6 or about 75% sequence identity with SEQ ID NO:6, a sequence having at least 80% sequence identity with SEQ ID NO:6 or about 80% sequence identity with SEQ ID NO:6, a sequence having at least 85% sequence identity with SEQ ID NO:6 or about 85% sequence identity with SEQ ID NO:6, a sequence having at least 90% sequence identity with SEQ ID NO:6 or about 90% sequence identity with SEQ ID NO:6, a sequence having at least 95% sequence identity with SEQ ID NO:6 or about 95% sequence identity with SEQ ID NO:6, a sequence having at least 96% sequence identity with SEQ ID NO:6, a sequence having at least 97% sequence identity with SEQ ID NO:6, a sequence having at least 98% sequence identity with SEQ ID NO:6, a sequence having at least 99% sequence identity with SEQ ID NO:6, JJB-B2 set forth as QVQLVETGGGLVQPGGSLRLSCAASESIFSTYAMGWYRQAPGKQRELVAAITTNDIA NYADSVKGRFTISRDNAKNTVYLQMNSLNPEDTAVYYCNAIFPPYNYWGQGTQVT VSSEPKTPKPQ (SEQ ID NO:7), a sequence having at least 70% sequence identity with SEQ ID NO:7 or about 70% sequence identity with SEQ ID NO:7, a sequence having at least 75% sequence identity with SEQ ID NO:7 or about 75% sequence identity with SEQ ID NO:7, a sequence having at least 80% sequence identity with SEQ ID NO:7 or about 80% sequence identity with SEQ ID NO:7, a sequence having at least 85% sequence identity with SEQ ID NO:7 or about 85% sequence identity with SEQ ID NO:7, a sequence having at least 90% sequence identity with SEQ ID NO:7 or about 90% sequence identity with SEQ ID NO:7, a sequence having at least 95% sequence identity with SEQ ID NO:7 or about 95% sequence identity with SEQ ID NO:7, a sequence having at least 96% sequence identity with SEQ ID NO:7, a sequence having at least 97% sequence identity with SEQ ID NO:7, a sequence having at least 98% sequence identity with SEQ ID NO:7, a sequence having at least 99% sequence identity with SEQ ID NO:7, JJB-B5 set forth as QVQLVETGGGLVQPGGSLRPSCTASGSIFSIYAMGWYRQASGKQRELVALITRDEVF NYADSVKGRFTISRDNAKDTVYLQMNSLKPEDTAVYYCWVETVNDHYNSGVEDY WGQGTQVTVSSEPKTPKPQ (SEQ ID NO:8), a sequence having at least 70% sequence identity with SEQ ID NO:8 or about 70% sequence identity with SEQ ID NO:8, a sequence having at least 75% sequence identity with SEQ ID NO:8 or about 75% sequence identity with SEQ ID NO:8, a sequence having at least 80% sequence identity with SEQ ID NO:8 or about 80% sequence identity with SEQ ID NO:8, a sequence having at least 85% sequence identity with SEQ ID NO:8 or about 85% sequence identity with SEQ ID NO:8, a sequence having at least 90% sequence identity with SEQ ID NO:8 or about 90% sequence identity with SEQ ID NO:8, a sequence having at least 95% sequence identity with SEQ ID NO:8 or about 95% sequence identity with SEQ ID NO:8, a sequence having at least 96% sequence identity with SEQ ID NO:8, a sequence having at least 97% sequence identity with SEQ ID NO:8, a sequence having at least 98% sequence identity with SEQ ID NO:8, a sequence having at least 99% sequence identity with SEQ ID NO:8, C17 set forth as EVQLVESGGGFVQAGESLTLSCTSSTLSCTSSTLTFTPYRMAWYRQAPGKQRDLVAD ISSGDGRTTNYADFAKGRFTISRDNIKNTVFLRMTNLKPEDTAVYYCNTFVSFGIAR S WGQGTQVTVSSEP (SEQ ID NO:9), a sequence having at least 70% sequence identity with SEQ ID NO:9 or about 70% sequence identity with SEQ ID NO:9, a sequence having at least 75% sequence identity with SEQ ID NO:9 or about 75% sequence identity with SEQ ID NO:9, a sequence having at least 80% sequence identity with SEQ ID NO:9 or about 80% sequence identity with SEQ ID NO:9, a sequence having at least 85% sequence identity with SEQ ID NO:9 or about 85% sequence identity with SEQ ID NO:9, a sequence having at least 90% sequence identity with SEQ ID NO:9 or about 90% sequence identity with SEQ ID NO:9, a sequence having at least 95% sequence identity with SEQ ID NO:9 or about 95% sequence identity with SEQ ID NO:9, a sequence having at least 96% sequence identity with SEQ ID NO:9, a sequence having at least 97% sequence identity with SEQ ID NO:9, a sequence having at least 98% sequence identity with SEQ ID NO:9, a sequence having at least 99% sequence identity with SEQ ID NO:9, JJB-D1 set forth as QVQLVESGGLVQAGGSLRPSCAASGSIFLQNAMGWYRQVPGKQRELVAAITSVDST NYADSVKGRFTISRDNAKNTVYLQMNSLKPEDTAVYYCNAPWNSDYHWGKGTLXVT VSSAHHSEDPS (SEQ ID NO:10), a sequence having at least 70% sequence identity with SEQ ID NO:10 or about 70% sequence identity with SEQ ID NO:10, a sequence having at least 75% sequence identity with SEQ ID NO:10 or about 75% sequence identity with SEQ ID NO:10, a sequence having at least 80% sequence identity with SEQ ID NO:10 or about 80% sequence identity with SEQ ID NO:10, a sequence having at least 85% sequence identity with SEQ ID NO:10 or about 85% sequence identity with. SEQ ID NO:10, a sequence having at least 90% sequence identity with SEQ ID NO:10 or about 90% sequence identity with SEQ ID NO:10, a sequence having at least 95% sequence identity with SEQ ID NO:10 or about 95% sequence identity with SEQ ID NO:10, a sequence having at least 96% sequence identity with SEQ ID NO:10, a sequence having at least 97% sequence identity with SEQ ID NO:10, a sequence having at least 98% sequence identity with SEQ ID NO:10, a sequence having at least 99% sequence identity with SEQ ID NO:10, VHH122 set forth as EVQLQESGGGLVQAGDSLRLSCLVSGRSFNSYTMGWFRQAPGKEREFVAAILWSGP TTYYADSVKGRFTISRDNAKNTVYLQMNSLKPEDTAVYYCAAALGVLVLAPGNVY SYWGQGTQVTVSS (SEQ ID NO:11) a sequence having at least 70% sequence identity with SEQ ID NO:11. or about 70% sequence identity with SEQ ID NO:11, a sequence having at least 75% sequence identity with SEQ ID NO:11 or about 75% sequence identity with SEQ ID NO:11, a sequence having at least 80% sequence identity with SEQ ID NO:11 or about 80% sequence identity with SEQ ID NO:11, a sequence having at least 85% sequence identity with SEQ ID NO:11 or about 85% sequence identity with SEQ ID NO:11, a sequence having at least 90% sequence identity with SEQ ID NO:11 or about 90% sequence identity with SEQ ID NO:11, a sequence having at least 95% sequence identity with SEQ ID NO:11 or about 95% sequence identity with SEQ ID NO:11, a sequence having at least 96% sequence identity with SEQ ID NO:11, a sequence having at least 97% sequence identity with SEQ ID NO:11, a sequence having at least 98% sequence identity with SEQ ID NO:11, and a sequence having at least 99% sequence identity with SEQ ID NO:11.

In various configurations, a third pseudo-repeat of an Ad5 fiber shaft domain, of a polypeptide of the present teachings can be joined to the carboxy-terminal portion of a T4fibritin protein sequence at a fragment of an insertion loop preceding a fifth coiled-coil. segment of a α-helical central domain of the fibritin.

In some embodiments, the present teachings include a nucleic acid encoding at least one polypeptide of the present teachings.

In some embodiments, the present teachings include an adenovirus vector comprising at least one polypeptide of the present teachings. In various configurations, adenovirus vector of the present teachings can further comprise a therapeutic gene.

In some embodiments, the present teachings include a method of treating a neoplastic disease in a subject. In some embodiments, the present teachings include a method of delivering a therapeutic adenovirus to a tumor cell. In some embodiments, the present teachings include a method of targeting a vector to CEA-expressing cells.

In some embodiments, the present teachings include methods of killing a tumor cell in a subject. These methods can comprise administering a therapeutically effective amount of a vector comprising a polypeptide of the present teachings. These methods can further comprise subjecting a subject to ionizing radiation in an amount effective for inducing CEA overexpression whereby the ionizing radiation enhances CEA-targeted Ad binding. In various configurations, a subject can be a mammal. In various configurations, a subject can be a human. In various configurations, a subject can have cancer. In various configurations, a cancer can be colon cancer, colorectal adenocarcinoma, rectal cancer, breast cancer, pancreatic cancer, prostate cancer, long cancer, or a combination thereof.

In various configurations, a method of administration can be, without limitation, intravenous administration, intraperitoneal administration, systemic administration, oral administration, intra tumoral administration, or a combination thereof.

In various embodiments, a polypeptide of the present teachings can comprise, consist essentially of, or consist of, in N-terminal-to-C-terminal order; an N-terminal segment of Ad5 fiber tall sequence, at least two pseudorepeats of an Ad5 fiber shaft domain sequence, a portion of a third Ad5 fiber shaft domain sequence, a carboxy-terminal segment of a T4fibritin bacteriophage trimerization domain sequence, a linker sequence and a camelid single chain antibody sequence. The carboxy-terminal segment of the T4 fibritin bacteriophage trimerization domain sequence can comprise an α-helical domain and a foldon domain. The N-terminal segment of Ad5 fiber tail sequence can be of sequence set forth as SEQ ID NO:1, a sequence having at least 70% sequence identity with SEQ ID NO:1 or about 70% sequence identity with SEQ ID NO:1, a sequence having at least 75% sequence identity with SEQ ID NO:1 or about 75% sequence identity with SEQ ID NO:1, a sequence having at least 80% sequence identity with SEQ ID NO:1 or about 80% sequence identity with SEQ ID NO:1, a sequence having at least 85% sequence identity with SEQ ID NO:1 or about 85% sequence identity with SEQ ID NO:1, a sequence having at least 90% sequence identity with SEQ ID NO:1 or about 90% sequence identity with SEQ ID NO:1, a sequence having at least 95% sequence identity with SEQ ID NO:1 or about 95% sequence identity with SEQ ID NO:1, a sequence having at least 96% sequence identity with SEQ ID NO:1, a sequence having at least 97% sequence identity with SEQ ID NO:1, a sequence having at least 98% sequence identity with SEQ ID NO:1, or a sequence having at least 99% sequence identity with SEQ ID NO:1.

In various configurations, the at least 2 pseudorepeats of an Ad5 fiber shaft domain sequence can be of sequence set forth as SEQ ID NO:2, a sequence having at least 70% sequence identity with SEQ ID NO:2 or about 70% sequence identity with SEQ ID NO:2, a sequence having at least 75% sequence identity with SEQ ID NO:2 or about 75% sequence identity with SEQ ID NO:2, a sequence having at least 80% sequence identity with SEQ ID NO:2 or about 80% sequence identity with SEQ ID NO:2, a sequence having at least 85% sequence identity with SEQ ID NO:2 or about 85% sequence identity with SEQ ID NO:2, a sequence having at least 90% sequence identity with SEQ ID NO:2 or about 90% sequence identity with SEQ ID NO:2, a sequence having at least 95% sequence identity with SEQ ID NO:2 or about 95% sequence identity with SEQ ID NO:2, a sequence having at least 96% sequence identity with SEQ ID NO:2, a sequence having at least 97% sequence identity with SEQ ID NO:2, a sequence having at least 98% sequence identity with SEQ ID NO:2, or a sequence having at least 99% sequence identity with SEQ ID NO:2. The portion of a third Ad5 fiber shaft domain sequence can be of sequence set forth as SEQ ID NO:3, a sequence having at least 70% sequence identity with SEQ ID NO:3 or about 70% sequence identity with SEQ ID NO:3, a sequence having at least 75% sequence identity with SEQ ID NO:3 or about 75% sequence identity with SEQ ID NO:3, a sequence having at least 80% sequence identity with SEQ ID NO:3 or about 80% sequence identity with SEQ ID NO:3, a sequence having at least 85% sequence identity with SEQ ID NO:3 or about 85% sequence identity with SEQ ID NO:3. The carboxy-terminal segment of a T4 fibritin bacteriophage trimerization domain sequence can be of sequence set forth as SEQ ID NO:4, a sequence having at least 70% sequence identity with SEQ ID NO:4 or about 70% sequence identity with SEQ ID NO:4, a sequence having at least 75% sequence identity with SEQ ID NO:4 or about 75% sequence identity with SEQ ID NO:4, a sequence having at least 80% sequence identity with SEQ ID NO:4 or about 80% sequence identity with SEQ ID NO:4, a sequence having at least 85% sequence identity with SEQ ID NO:4 or about 85% sequence identity with SEQ ID NO:4, a sequence having at least 90% sequence identity with SEQ ID NO:4 or about 90% sequence identity with SEQ ID NO:4, a sequence having at least 95% sequence identity with. SEQ ID NO:4 or about 95% sequence identity with SEQ ID NO:4, a sequence having at least 96% sequence identity with SEQ ID NO:4, a sequence having at least 97% sequence identity with SEQ ID NO:4, a sequence having at least 98% sequence identity with SEQ ID NO:4, or a sequence having at least 99% sequence identity with SEQ ID NO:4. The Sinker sequence can comprise or consist of the sequence (GlyₙSer)ₘ where n is an integer from 2 to 6 , and m is an integer from 1 to 5. The peptide linker can also be Gly-Gly-Gly-Gly-Ser (SEQ ID NO:5).

In various configurations, the camelid single chain antibody sequence can be against a human carcinoembryonic antigen. The camelid single chain antibody sequence can be selected from the group consisting of JJB-A3 set forth as SEQ ID NO:6, a sequence having at least 70% sequence identity with SEQ ID NO:6 or about 70% sequence identity with SEQ ID NO:6, a sequence having at least 75% sequence identity with SEQ ID NO:6 or about 75% sequence identity with SEQ ID NO:6, a sequence having at least 80% sequence identity with SEQ ID NO:6 or about 80% sequence identity with SEQ ID NO:6, a sequence having at least 85% sequence identity with SEQ ID NO:6 or about 85% sequence identity with SEQ ID NO:6, a sequence having at least 90% sequence Identity with SEQ ID NO:6 or about 90% sequence identity with SEQ ID NO:6, a sequence having at least 95% sequence identity with SEQ ID NO:6 or about 95% sequence identity with SEQ ID NO:6, a sequence having at least 96% sequence identity with SEQ ID NO:6, a sequence having at least 97% sequence identity with SEQ ID NO:6, a sequence having at least 98% sequence identity with SEQ ID NO:6, a sequence having at least 99% sequence identity with SEQ ID NO:6, JJB-B2 set forth as SEQ ID NO:7, a sequence having at least 70% sequence identity with SEQ ID NO:7 or about 70% sequence identity with SEQ ID NO:7, a sequence having at least 75% sequence identity with SEQ ID NO:7 or about 75% sequence identity with SEQ ID NO:7, a sequence having at least 80% sequence identity with SEQ ID NO:7 or about 80% sequence identity with SEQ ID NO:7, a sequence having at least 85% sequence identity with SEQ ID NO:7 or about 85% sequence identity with SEQ ID NO:7, a sequence having at least 90% sequence identity with SEQ ID NO:7 or about 90% sequence identity with SEQ ID NO:7, a sequence having at least 95% sequence identity with SEQ ID NO:7 or about 95% sequence identity with SEQ ID NO:7, a sequence having at least 96% sequence identity with SEQ ID NO:7, a sequence having at least 97% sequence identity with SEQ ID NO:7, a sequence having at least 98% sequence identity with SEQ ID NO:7, a sequence having at least 99% sequence identity with SEQ ID NO:7, JJB-B5 set forth as SEQ ID NO:8, a sequence having at least 70% sequence identity with. SEQ ID NO:8 or about 70% sequence identity with SEQ ID NO:8, a sequence having at least 75% sequence identity with SEQ ID NO:8 or about 75% sequence identity with SEQ ID NO:8, a sequence having at least 80% sequence identity with SEQ ID NO:8 or about 80% sequence identity with SEQ ID NO:8, a sequence having at least 85% sequence identity with SEQ ID NO:8 or about 85% sequence identity with SEQ ID NO:8, a sequence having at least 90% sequence identity with SEQ ID NO:8 or about 90% sequence identity with SEQ ID NO:8, a sequence having at least 95% sequence Identity with SEQ ID NO:8 or about 95% sequence identity with SEQ ID NO:8, a sequence having at least 96% sequence identity with SEQ ID NO:8, a sequence having at least 97% sequence identity with SEQ ID NO:8, a sequence having at least 98% sequence identity with SEQ ID NO:8, a sequence having at least 99% sequence Identity with SEQ ID NO:8, C17 set forth as (SEQ ID NO:9), a sequence having at least 70% sequence identity with SEQ ID NO:9 or about 70% sequence identity with SEQ ID NO:9, a sequence having at least 75% sequence identity with SEQ ID NO:9 or about 75% sequence identity with SEQ ID NO:9, a sequence having at least 80% sequence identity with SEQ ID NO:9 or about 80% sequence identity with SEQ ID NO:9, a sequence having at least 85% sequence identity with SEQ ID NO:9 or about 85% sequence identity with SEQ ID NO:9, a sequence having at least 90% sequence identity with SEQ ID NO:9 or about 90% sequence identity with SEQ ID NO:9, a sequence having at least 95% sequence identity with SEQ ID NO:9 or about 95% sequence identity with SEQ ID NO:9, a sequence having at least 96% sequence identity with SEQ ID NO:9, a sequence having at least 97% sequence identity with SEQ ID NO:9, a sequence having at least 98% sequence identity with SEQ ID NO:9, a sequence having at least 99% sequence identity with SEQ ID NO:9, JJB-D1 set forth as SEQ ID NO:10, a sequence having at least 70% sequence identity with SEQ ID NO:10 or about 70% sequence identity with SEQ ID NO:10, a sequence having at least 75% sequence identity with. SEQ ID NO:10 or about 75% sequence identity with SEQ ID NO:10, a sequence having at least 80% sequence identity with SEQ ID NO:10 or about 80% sequence identity with SEQ ID NO: 10, a sequence having at least 85% sequence identity with SEQ ID NO:10 or about 85% sequence identity with SEQ ID NO:10, a sequence having at least 90% sequence identity with SEQ ID NO:10 or about 90% sequence identity with SEQ ID NO:10, a sequence having at least 95% sequence identity with SEQ ID NO:10 or about 95% sequence identity with SEQ ID NO:10, a sequence having at least 96% sequence identity with SEQ ID NO:10, a sequence having at least 97% sequence identity with SEQ ID NO:10, a sequence having at least 98% sequence identity with SEQ ID NO:10, a sequence having at least 99% sequence identity with SEQ ID NO:10, VHH 122 set forth as EVQLQESGGGLVQAGDSLRLSCLVSGRSFNSYTMGWFRQAPGKEREFVAAILWSGP TTYYADSVKGRFTISRDNAKNTVYLQMNSLKPEDTAVYYCAAALGVLVLAPGNVY SYWGQGTQVTVSS (SEQ ID NO:11) a sequence having at least 70% sequence identity with SEQ ID NO:11 or about 70% sequence identity with SEQ ID NO:11, a sequence having at least 75% sequence identity with SEQ ID NO:11 or about 75% sequence identity with SEQ ID NO:11, a sequence having at least 80% sequence identity with SEQ ID NO:11 or about 80% sequence identity with SEQ ID NO:11, a sequence having at least 85% sequence identity with SEQ ID NO:11 or about 85% sequence identity with SEQ ID NO:11, a sequence having at least 90% sequence identity with SEQ ID NO:11 or about 90% sequence identity with SEQ ID NO:11, a sequence having at least 95% sequence identity with SEQ ID NO:11 or about 95% sequence identity with SEQ ID NO:11, a sequence having at least 96% sequence identity with SEQ ID NO:11, a sequence having at least 97% sequence identity with SEQ ID NO:1, a sequence having at least 98% sequence identity with SEQ ID NO:11, and a sequence having at least 99% sequence identity with SEQ ID NO:11.

In various configurations, the camelid single chain antibody can be anti-hCEA VHH (VHH122) set forth as SEQ ID NO:11, a sequence having at least 70% sequence identity with SEQ ID NO:11 or about 70% sequence identity with SEQ ID NO:11, a sequence having at least 75% sequence identity with SEQ ID NO:11 or about 75% sequence identity with SEQ ID NO:11, a sequence having at least 80% sequence identity with SEQ ID NO:11 or about 80% sequence identity with SEQ ID NO:11, a sequence having at least 85% sequence identity with SEQ ID NO:11 or about 85% sequence identity with SEQ ID NO:11, a sequence having at least 90% sequence identity with SEQ ID NO:11 or about 90% sequence identity with SEQ ID NO:11, a sequence having at least 95% sequence identity with SEQ ID NO:11 or about 95% sequence identity with SEQ ID NO:11, a sequence having at least 96% sequence identity with SEQ ID NO:11, a sequence having at least 97% sequence identity with SEQ ID NO:11, a sequence having at least 98% sequence identity with SEQ ID NO:11, or a sequence having at least 99% sequence identity with SEQ ID NO:11.

In various configurations, a third pseudo-repeat of the Ad5 fiber shaft domain can be joined to the carboxy-terminal portion of a T4 fibritin protein sequence at a fragment of an insertion loop preceding a fifth coiled-coil segment of a α-helical central domain of the fibritin.

In various configurations, a nucleic acid encoding a polypeptide can comprise, consist essentially of, or consist of, a nucleic acid sequence encoding a polypeptide in N-terminal-to-C-terminal order: an N-terminal segment of Ad5 fiber tail sequence, at least two pseudorepeats of an Ad5 fiber shaft domain sequence, a portion of a third Ad5 fiber shaft domain sequence, a carboxy-terminal segment of a T4 fibritin bacteriophage trimerization domain, sequence, a linker sequence and a camelid single chain antibody sequence. In various configurations, an adenovirus vector can comprise a polypeptide comprising, consisting essentially of, or consisting of, in N-terminal-to-C-terminal order: an N-terminal segment of Ad5 fiber tail sequence, at least two pseudorepeats of an Ad5 fiber shaft domain sequence, a portion of a third Ad5 fiber shaft domain sequence, a carboxy-terminal segment of a T4 fibritin bacteriophage trimerization domain sequence, a linker sequence and a camelid single chain antibody sequence. In various configurations, an adenovirus can further comprise a therapeutic gene.

In various embodiments, a method of treating a neoplastic disease in a subject can comprise administering a therapeutically effective amount of a vector comprising a polypeptide of the present teachings. In some configurations, a method of treating a neoplastic disease in a subject can comprise administering a therapeutically effective amount of a vector comprising a polypeptide in accordance with the present teachings. In some configurations, a method of delivering a therapeutic adenovirus to a tumor cell can comprise administering to a subject a therapeutically effective amount of a vector comprising a polypeptide in accordance with the present teachings. In various configurations, a method of delivering a therapeutic adenovirus to a tumor cell can comprise administering to a subject a therapeutically effective amount of a vector comprising a polypeptide in accordance with the present teachings.

In various embodiments, a method of targeting a vector to CEA-expressing cells can comprise administering to a subject a vector comprising a polypeptide in accordance with the present teachings. In various configurations, a method of killing a tumor cell in a subject can comprise administering to a subject a therapeutically effective amount of a vector comprising a polypeptide in accordance with the present teachings. In various aspects, a method of killing a tumor cell In a subject can comprise administering to a subject a therapeutically effective amount of a vector comprising a polypeptide in accordance with the present teachings.

In various embodiments, methods of treating disease utilizing a vector of the present teachings can further comprise subjecting the subject to ionizing radiation in an amount effective for inducing CEA overexpression whereby the ionizing radiation enhances CEA-targeted Ad binding.

In various methods of the present teachings, the subject can be a mammal or a human. The subject can have cancer. The cancer can be, for example and without limitation, colon cancer, colorectal adenocarcinoma, rectal cancer, breast cancer, pancreatic cancer, prostate cancer, lung cancer, or a combination thereof.

In methods of the present teachings, a method of administration can be, for example and without limitation, intravenous administration, intraperitoneal administration, systemic administration, oral administration, intratumoral administration, or a combination thereof.

### BRIEF DESCRIPTION OF THE DRAWINGS

FIG. 1 illustrates a multiple amino acid sequence alignment of camelid VHH clones.

FIG. 2A-D illustrate an evaluation of anti-CEA VHH binding to hCEA protein.

FIG. 3A-B illustrate evaluation of FF-VHH expressing Ad vectors. FIG. 3A illustrates a simplified schematic of recombinant Ad vector genomes with indicated regions highlighted.

FIG. 3B illustrates an assessment of incorporation of FF-VHH proteins into Ad particles using Western blotting analysis.

FIG. 4A-C illustrate an initial screening of binding properties of the recombinant Ad vectors. FIG. 4 A illustrates evaluation of Ad vectors binding to hCEA protein by using ELISA. FIG. 4B illustrates level of hCEA mRNA expression determined by reverse transcriptase polymerase chain reaction (RT-PCR). FIG. 4C illustrates evaluation of the specificity of AdB2Luc-mediated gene transfer.

FIGS. 5A-D illustrate evaluation of efficacy and specificity of the CEA-targeted gene transfer. FIG. 5 A illustrates hCEA expression. Evaluation of the efficacy of Ad-mediated reporter gene transfer: MC38 (FIG. 5B) and MC38CEA (FIG. 5C) FIG. 5D illustrates Ad targeting efficiency.

FIG. 6A-G illustrate AdB2Luc displaying an anti-hCEA VHH produces CAR-independent and CEA-dependent gene transfer. FIG. 6A illustrates hCAR expression CHO and CHO-CAR Chinese hamster ovary cells subjected to FACS analysis. FIG. 6B illustrates CHO (hCAR−) and CHO-CAR (hCAR+) cells pre-incubated with soluble Ad5 knob protein at different concentration and infected with 5×10³ v.p. per cell of AdB2Luc. FIG. 6C illustrates relative Luc expression following infection with AdB2Luc. FIG. 6D illustrates inhibition of Ad5 Luc-mediated gene transfer. FIG. 6E illustrates relative Luc expression following infection with Ad5 Luc. FIG. 6F illustrates inhibition of AdB2Luc-mediated gene transfer. AdB2Luc was pre-incubated with hCEA or BSA at different concentration. FIG. 6G illustrates relative Luc expression following infection with AdB2Luc.

FIG. 7A-E illustrate radiation treatment of cancer cells increasing AdB2Luc infection.

FIG. 8A-B illustrate validation of incorporation of sdAb-targeted chimeric fiber protein in CRAds.

FIG. 9 illustrates the binding specificity of sdAb-targeted CRAds.

FIG. 10A-C illustrate in vitro characterization of CRAd replication.

FIG. 11 illustrates that hCEA-targeted CRAd specifically kills hCEA positive tumor cells and mitigates off target cytotoxicity.

FIG. 12 illustrates that hCEA-targeted CRAd does not kill immortalized normal liver cells.

FIG. 13A-B illustrate Ad.CXCR4E1.B2 induces hCEA-dependent and hCAR-independent oncolysis.

FIG. 14 illustrates transduction of murine DC line DC2.4 by Nb-DC1.8-targeted Ad vector.

FIG. 15A-B illustrate transduction of immature BMDCs by Nb-DC 1.8 targeted ad vector in vitro.

### DETAILED DESCRIPTION

The inventors disclose Ad vectors modified to comprise anti-CEA VHH in the fiber protein for cell-selective transgene expression. In some configurations, Ad vectors disclosed herein include fiber modifications including VHH. In various aspects, the introduction of a VHH can facilitate tumor-selective recombinant Ad transduction. The inventors demonstrate that at least one anti-hCEA VHH can retain antigen recognition functionality and can provide specificity of gene transfer of capsid-modified Ad5 vector.

In some embodiments to develop CEA-targeted recombinant Ad5-based vectors, the inventors genetically incorporated anti-hCEA VHH into a de-knobbed Ad5 fiber-fibritin protein. The inventors demonstrated that the modified vector retained trimerization capability of Ad fiber as well as antigen recognition functionality of anti-hCEA VHH. The inventors demonstrated the ability of anti-CEA VHH fused to fiber-fibritin chimera to provide specific and efficient targeted Ad-mediated gene transfer to CEA-expressing cancer cells. In some embodiments, deletion of the knob can reduce binding of the vector to undesired targets.

The inventors investigated whether binding specificity of some of the VHHs would be altered due to the relatively larger size of a modified chimeric VHH-FF protein. Results demonstrated selective targeting of modified Ad vectors to the cognate epitope expressed on the surface ELISA plate as well as on the membrane of cancer cells (see Examples). Additionally, results of competitive inhibition studies confirmed CEA-dependent and CAR-independent AdB2Luc-mediated, gene transduction (see Examples).

The inventors derived a VHH-incorporating Ad5 vector which demonstrates targeting to CEA expressing cells dictated by the embodied VHH.

Administration can be by any administration route known to skilled artisans. In some embodiments, representative routes of administration include, without limitation, intravenously, intraperitoneally, systemically, orally and intratumorally.

### Abbreviations

### Ab: Antibody

### Ad: Adenovirus

Ad5: Adenovirus serotype 5
BMDCs: Bone marrow dendritic cells.
BSA: Bovine serum albumin
CAR: Coxsackie and adenovirus receptor
CEA: Carcinoembryonic antigen
CHO: Chinese hamster ovary

### CMV: Cytomegalovirus

CRAds: conditionally replicative adenoviruses
EC₅₀: Half maximal effective concentration
EGFR: Epidermal growth factor receptor
FACS: Fluorescence-activated cell sorting
FBS: Fetal bovine serum

### FF: Fiber-fibritin

HEK: Human embryonic kidney
HRP: Horseradish peroxidase

### Ig: Immunoglobulin

### Luc: Luciferase

mAb: Monoclonal antibody
ORF: Open reading frame
PC: Prostate cancer
PCR: Polymerase chain reaction
PI: Propidium iodide
PVDF: Polyvinylidene difluoride
qPCR: Quantitative polymerase chain reaction
RLU: Relative light units
RT-PCR: Reverse transcriptase polymerase chain reaction
s.d.: Standard deviation
seFv; Single-chain variable fragment
sdAb: Single domain antibodies
TBS: Tris-buffered saline
VHH: Variable heavy domain
v.p/vp: Viral particles

### Methods

The methods and compositions described herein utilize laboratory techniques well known to skilled artisans, and can be found in laboratory manuals such as Sambrook, J,, et al., Molecular Cloning: A Laboratory Manual, 3rd ed. Cold Spring Harbor Laboratory Press, Cold Spring Harbor, N.Y., 2001; Spector, D. L. et al., Cells: A. Laboratory Manual, Cold Spring Harbor Laboratory Press, Cold Spring Harbor, N.Y., 1998; Nagy, A., Manipulating the Mouse Embryo: A. Laboratory Manual (Third Edition), Cold Spring Harbor, N.Y., 2003 and Harlow, E., Using Antibodies: A Laboratory Manual, Cold Spring Harbor Laboratory Press, Cold Spring Harbor, N.Y., 1999. Methods of administration of pharmaceuticals and dosage regimes, can be determined according to standard principles of pharmacology well known skilled artisans, using methods provided by standard reference texts such as Remington: the Science and Practice of Pharmacy (Alfonso R. Gennaro ed. 19th ed. 1995); Hardman, J. G., et al., Goodman & Gilman's The Pharmacological Basis of Therapeutics, Ninth Edition, McGraw-Hill, 1996; and Rowe, R. C., et al., Handbook of Pharmaceutical Excipients, Fourth Edition, Pharmaceutical Press, 2003. As used in the present description and the appended claims, the singular forms “a”, “an” and “the” are intended to include the plural forms as well, unless the context indicates otherwise.

Immunization of Alpacas with CEA Protein

Purified human carcinoembryonic antigen (hCEA) protein (ProNique Scientific, Castle Rock, Colo.) was used to immunize alpacas in alum/CpG adjuvant as described in Maass, D. R., et al. 2007 Journal of Immunological Methods 324, 13-25. Two adult male alpacas were given six immunizations at three-week intervals, each including multi-site subcutaneous injections containing a total of 100 μg of hCEA in the pre-scapular region. Serum at the completion of the immunization process contained Ab titers for hCEA exceeding 1:10,000 in both alpacas.

### Identification of Anti-CEA VHHs

A VHH-display library was prepared from B cells obtained from the alpacas four days following the final boost with hCEA. A single VHH-display phage library was prepared using RNA from both alpacas. Library construction, panning, phage recovery and clone fingerprinting were performed as described (Maass, D. R., et al. Int. J. Parasitol 37, 953-962, 2007; Mukherjee, J., et al. PLoS ONE 7 e29941, 2012; Tremblay, J. M., et al., Infect. Immun. 81, 4592-4603, 2013). Approximately 6×10⁶ independent clones were obtained and pooled to yield the VHH-display phage library. The hCEA protein was coated onto Nunc Immunotubes (Nunc, Rochester, N.Y.) for panning. Following two panning cycles, >80% of the selected clones recognized hCEA on enzyme-linked immunosorbent assay (ELISA) (4-fold exceeding over background). The 38 clones producing the strongest signals were characterized by DNA fingerprinting as described, by Tremblay, J. M., et al. Infect Immun. 81, 4592-4603, 2013 and the inventors identified nine unique VHHs. DNA sequencing of these clones identified four hCEA-binding VHH families that, without being limited by theory, appeared unrelated. VHH representatives of the four families (JJB-A3 (SEQ ID NO:6), JJB-B2 (SEQ ID NO:7), JJB-B5 (SEQ ID NO:8) and JJB-D1 (SEQ ID NO:10)) were expressed as thioredoxin fusion proteins as described by Tremblay, J. M., et al., Toxicon, 56, 990-998, 2010, purified, and further characterized.

Dilution ELISAs were performed to assess the apparent affinity (EC₅₀) of each purified VHH as described by Mukherjee, J., et al. PLoS One., 7 e29941, 2012. Nunc Maxisorb plates (Nunc) were coated overnight at 4° C. with 1 μg/ml human CEA protein (Abeam, Cambridge, Mass.). The plates were blocked in binding buffer containing 5% w/v non-fat milk in Tris-buffered saline (TBS). The blocking buffer was replaced with a dilution series of either JJB-A3, JJB-B2, JJB-B5, or JJB-D1 in binding buffer with 0.05% Tween 20. Plates were incubated at 25° C. for one hour and then washed three times with TBS. Bound VHHs were detected with HRP/anti-E-tag mAb (Bethyl Laboratories, Montgomery, Tex.).

### Cells and Reagents

MC38CEA cells expressing hCEA were generated by retroviral transduction with CEA cDNA, The human embryonic kidney HEK293 cells were purchased from Microbix Biosystems (Ontario, Canada). Human colorectal adenocarcinoma LS174T cells, prostate adenocarcinoma PC-3 cells, lung cancer A549 and H460 cells were obtained from ATCC (Manassas, Va.). All cells were cultured in DMEM/F12 (Mediatech, Herndon, Va.) containing 10% fetal bovine serum (FBS) (Summit Biotechnology, Fort Collins, Colo.) and cultured at 37° C. in a humidified atmosphere with 5% CO₂. Anti-hCEA VHH clone C17 (SEQ ID NO:9) was obtained from a semi-synthetic camelid VHH phage library.

Human colorectal adenocarcinoma LS174T and human glioma U118MG cells were purchased from ATCC (Manassas, Va.). Human pancreatic carcinoma HS766T cells were kindly provided by Dr. PG Oliver (University of Alabama at Birmingham, Birmingham, Ala.). Human glioma U118-hCAR cells expressing hCAR were kindly provided by Dr J T Douglas (University of Alabama at Birmingham). For propagation of our vector we used HEK293 cells and 293F28 cells expressing wild-type Ad5 fiber protein, which have been described previously (Belousova, N., et al., J. Virol., 77,11367-11377, 2003). All above mentioned cell lines were cultured in DMEM/F12 (Mediatech, Nerndon, Va.) medium supplemented with 10% FBS, 100 IU/ml penicillin and 100 μg/ml streptomycin.

Immortalized primary human liver THLE-3 cells were purchased from ATCC and cultured in accordance with vendor instructions.

### Adenoviral Vectors

Replication incompetent E1-deleted Ad5 vectors were created using a two-plasmid rescue method. The chimeric fiber-fibritin (FF) protein containing the N-terminal Ad5 fiber tail region fused to the entire fibritin protein with the trimerizing foldon domain of bacteriophage T4 following by Gly-Gly-Gly-Gly-Ser (SEQ ID NO:5) peptide linker connected to the VHH open reading frame (ORF) as described by Noureddini, S. C., et al., Virus Res., 116,185-195, 2006. To generate a PCR product encoding a fragment of the VHH ORF clone B2: BamH1-B2 (TTA GGA TCC CAG GTG CAG CTC GTG) (SEQ ID NO:12) and B2-Swa1 (GGG ATT TAA ATA ATT GTG GTT TTG GTG) (SEQ ID NO:13): for clone C17: BamH1-C17 (AAA GGA TCC GAA GTC CAA CTG GTT G) (SEQ ID NO:14) and C17-Swa1 (TTT ATT TAA ATC AGG CCG CCG ACG A) (SEQ ID NO:15); clone VHH122: BamH1-VHH122 (AGA GGA TCC GAG GTG CAA CTG C) (SEQ ID NO:16) and VHH122-Swa1 (CCC ATT TAA ATC ATG AGG AGA CGG TG) (SEQ ID NO:17) primers were used.

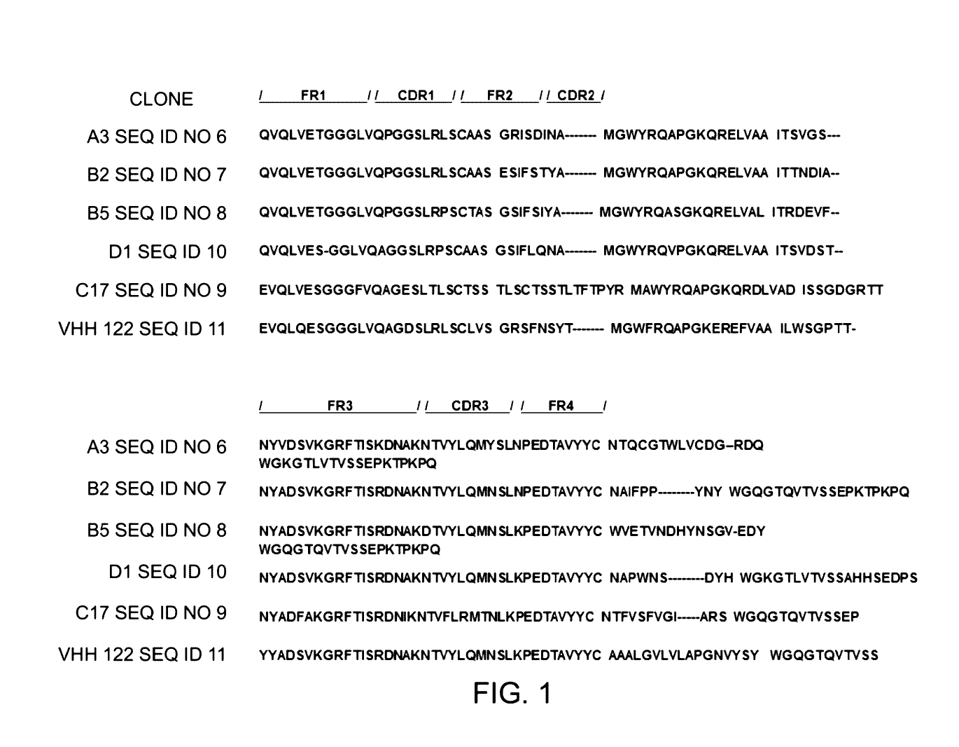

The PCR product was cloned into a plasmid pKan556FF using BamH I and Swa I sites to generate the pKan566FF-B2, pKan566FF-C17 and pKan566FF-VHH122, respectively. Insertion sequences were confirmed by using restriction enzyme mapping and partial sequence analysis. Predicted amino acid sequences of VHH domain of a camelid heavy chain Abs used in this study are summarized in FIG. 1. Sequences continue from the top panel to the bottom panel A3 is set forth in SEQ ID. NO.6, B2 is set forth in SEQ ID. NO 7. B5 is set forth in SEQ ID. NO.8. D1 is set forth in SEQ ID. NO.10, C17 is set forth in SEQ ID. NO.9. VHH122 is set forth in SEQ ID NO.11. Dashes indicate gaps introduced in order to optimize sequence alignment. VHH domain of a camelid heavy chain Abs clones B2 ( SEQ ID NO.7), C17 (SEQ ID. NO.9) and VHH122 (SEQ ID. NO.11) were used for genetic incorporation into the chimeric VHH-fiber-fibritin. Predicted molecular weight (MW) of VHHs: A3 (SEQ ID. NO.6): MW 13.8 kDa; B2 (SEQ ID. NO.7): MW 13.4 kDa; B5 (SEQ ID. NO.8): MW 14.4 kDa; D1: MW 13.2 kDa (SEQ ID NO.10); C17 (SEQ ID. NO.9): MW 14.1 kDa; VHH122 (SEQ ID NO.11): MW 13.5 kDa. FR1-4, framework regions; CDR1-3, complementarity determining regions.

The shuttle plasmids were linearized with Pme I enzyme and integrated into the Ad5 genome by homologous recombination in the E. coli strain BJ5183 with pVK700 plasmid comprised of the human cytomegalovirus (CMV) major immediate-early enhancer/promoter element coupled to the firefly luciferase (Luc) gene. The recombinant viral genomes with FF-VHH fusions were linearized with Pac I and then transfected into 293F28 cells using SuperFect® Transfection Reagent (Qiagen, Chatsworth, Calif.), where they were packaged into virus particles. 293F28 cells stably express the native Ad5 fiber, thus viruses rescued at this point were mosaic in the sense that the Ad5 virions randomly incorporated a mixture of native Ad5 fibers and FF-VHH chimeras. After additional round of amplification on 293F28 cells, the viruses were amplified in HEK293 cells, which do not express native Ad5 fiber, to obtain virus particles containing only FF-VHH proteins. To verify inserted modifications of the fiber gene all viral genomes were subjected to partial sequencing analysis. Viruses were propagated in HEK293 cells and purified twice by CsCI gradient centrifugation and dialyzed against 10 mM HEPES, 1 mM MgCl₂, pH 7.8 with 10% glycerol, as previously described by He, T. C., et al. 1998 PNAS 95(5): p. 2509-14. The concentration of viral particles (v.p.) was determined by measuring absorbance of the dissociated virus at A₂₆₀ nm using a conversion factor of 1.1×10¹² vp per absorbance unit. Multiplicity of infection for subsequent experiments was expressed as v.p. per cell.

### Enzyme-linked Immunosorbent Assay (ELISA)

Nunc Maxisorb® plates (Nunc) were coated overnight at 4° C. with human CEA protein (Abeam) diluted at a concentration of 1 μg/ml in 50 mM carbonate buffer (pH 8.6). The unsaturated surface of the wells was then blocked for 1 hour at 25°C. by the addition of 200 μl of blocking buffer including Tris-buffered saline (TBS) with 5% w/v non-fat milk (LabScientific, Livingston, N.J.). The blocking buffer was replaced with 100 μl of Ad diluted in binding buffer (TBS with 0.05% Tween 20 and 5% w/v non-fat milk). Plates were incubated at 25° C. for one hour and then washed three times with washing buffer (TBS with 0.05% Tween 20). Bound viral particles were detected by incubation for one hour at 25° C. with polyclonal anti-adenovirus goat Ab (ViroStat, Portland, Me.). The wells were washed three times with washing buffer and then anti-goat rabbit IgG conjugated with horseradish peroxidase (HRP) (Dako Corporation, Glostrup, Denmark) were added and incubation was continued for one hour. The color was developed with Sigma FAST o-phenylenediamine dihydrochloride (Sigma) as recommended by the manufacturer.

### RNA Preparation and RT-PCR Assay

The levels of hCEA mRNA expression in cells were determined by reverse transcriptase polymerase chain reaction (RT-PCR). Total RNA was extracted from 1×10⁷ cells using RNeasy® Mini Kit (Qiagen), following standard protocol, and quantified spectrophotometrically using a MBA 2000 spectrophotometer (Perkin Elmer, Wellesley, Mass.). The first-strand cDNA was synthesized using random hexamer primers and an Omniscript® RT kit (Qiagen) and used as the template for PGR. The following primers were used: hCEAf206: 5′-CCA CCA CTG CCA AGC TCA CTA-3′ (SEQ ID NO:18); hCEAr388: 5′-CTG GGG TAG CTT GTT GAG TTC CTA-3′ (SEQ ID NO:19) (amplicon 183 bp). After the initial denaturation (5 min at 95° C.), amplification was performed with 30 cycles of 30 sec at 95° C. 20 see at 62° C. and 35 sec 72° C., The hCEA gene specific qPCR template standard (OriGene Technologies, Rockville, Md.) was used as an internal standard for template loading. PCR products were analyzed by 1% agarose electrophoresis with ethidium bromide staining.

### Gene Transfer

Cells were seeded, at 1×10⁵ cells per well in 24-well tissue culture plates and allowed to grow overnight. The next day, cells were washed one time with PBS, and then infected with 5×10³ v.p. per cell of Ad vectors in triplicate. After one hour, cell culture media was removed, cells were washed with PBS and fresh media was added. Forty-eight hours afterward, cell culture media was removed, cells were washed one time with PBS, and cells were lysed and Luc activity was analyzed as described below.

### Expression of Recombinant Ad5 Knob

The knob domain, of Ad5 fiber protein was expressed in E. coli as described by Krasnykh, V. N., et al., J. Virol., 70, 6839-6846, 1996. Soluble His-tagged Ad5 knob was purified by gravity-flow affinity chromatography using a Ni-NTA resin (Qiagen). The concentration of the purified protein was determined using DC Protein Assay (Bio-Rad, Hercules, Calif.), according to the manufacturer's instructions. Purified recombinant protein was evaluated by Western blot using anti-His mAb (Sigma).

### Competitive Inhibition of Gene Transfer

Cells were seeded at 1×10⁵ cells per well in 24-well tissue culture plates and allowed to grow overnight. The next day, cells were washed one time with PBS. and incubated for one hour at 37° C. with serial dilutions of Ad5 fiber knob protein or BSA. For hCEA mediated inhibition of gene transfer Ad5 were preincubated with hCEA or BSA at different concentration at 37° C. for 1 hour. Then, cells were washed one time with PBS, and infected with Ad5 at 5×10³ vp per cell Alter incubation for one hour at 37° C. cell culture media was removed, cells were washed with PBS and fresh media was added. Forty-eight hours afterward, cell culture media was removed, cells were washed one time with PBS, and cells were lysed and Luc activity was analyzed (See Methods below).

### Luciferase Assay

The Luciferase Assay System (Promega) and ORION microplate luminometer (Berthold Detection systems, Oak Ridge, Tenn.) were used for the evaluation of Luc activity of infected cells. Luciferase activity was normalized by the protein concentration of the cell lysate using DC Protein Assay (Bio-Rad), according to the manufacturer's instructions. Data are expressed as relative light units (RLU) per 1×10⁴ cells and bars represent the mean±the standard deviation (s.d.).

### Western Blotting

Samples were preincubated in Laemmli sample buffer at 95° C. for five minutes and separated using a 4-20% gradient polyacrylamide gel (Bio-Rad). For electrophoresis under semi-native condition, samples were not boiled. The proteins were electroblotted onto polyvinylidene difluoride (PVDF) membranes and the blots were developed with SIGMA FAST™ 3,3′-diaminobenzidime system (Sigma) according to the manufacturer's protocol using anti-Ad5 fiber tail mAb 402 as the primary antibody.

### Real-time Quantitative PCR

Quantitative analysis of the Ad5 hexon gene expression was performed rising real-time PCR. For in vitro studies, human cancer PC-3, LS174T and A549 cells were plated into six-well tissue culture plates at 3×10⁵ cells per well, and allowed to adhere overnight. Next day, cells were either mock- irradiated or irradiated at 6 Gy using the RS-2000 Biological System X-ray irradiator (Rad Source Technologies, Suwannee, Ga.). Twenty-four hours later, the cells were infected with AdB2Luc or Ad5Luc at 5×10 ³ vp per cell. After incubation for one hour at 37° C. cell culture media was removed, cells were washed one time with PBS, collected, and total DNA was extracted using QIAAMP* DNA Mini Kit (QIAGEN).

For preparation of control samples, AdB2Luc genomic DNA was extracted from purified viral stock by using a QIAAMP® DNA Mini Kit. Serial 10-fold dilutions (from 1×10⁹ to 10 viral particles per reaction) of viral DNA were included in each run to establish a standard curve for quantitative appraisal of hexon gene copy number. For detection of the Ad hexon gene, the following primers and TAQMAN® probe were used: Ad5Hexon-fwd: 5′-TAC GCA CGA CGT GAC CAC A-3′ (SEQ ID NO:20), Ad5Hexon-rev: 5′-ATC CTC ACG GTC CAC AGG G-3′ (SEQ ID NO:21) and Ad5Hexon-probe: 5′-6FAM-ACC GGT CCC AGC GTT TGA CGC-BHQ-1-3′ (SEQ ID NO:22): for human β-Actin gene expression: β-Actin-fwd: 5′-GAG GCA TCC TCA CCC TGA AG-3′ (SEQ ID NO:23), β-Actin-rev: 5′-TCC ATG TCG TCC CAG TTG GT-3′ (SEQ ID NO:24), and β-Actin-probe: 5′-HEX-CCC CAT CGA GCA CGG CAT CG-BHQ1-3′ (SEQ ID NO:25). In each reaction, 20 ng of total DNA was used as template and PCR was performed in 25 μl of reaction mixture containing 12.5 μl of 2× TAQMAN® Universal PCR master Mix (PE Applied Biosystems, Foster City, Calif.), 300 nM each primer, and 100 nM fluorogenic probe. Amplifications were carried out in a 96-well reaction plate (PE Applied Biosystems) in a spectrofluorimetric thermal cycler (ABI PRISM® 7000 Sequence Detector; PE Applied Biosystems). After the initial denaturation (2 min at 95° C.), amplification was performed with 45 cycles of 15 seconds at 95° C. and 60 seconds at 60° C. Each sample was run in triplicate. A threshold cycle (Cₜ) for each triplicate was estimated by determining the point at which the fluorescence exceeded a threshold limit (10-fold the standard deviation of the baseline). Level of the AdB2Luc and Ad5Luc binding in human cancer cells was determined as the Ad hexon gene copy number per 1 ng total DNA.

Fluorescence-activated Cell Sorter (FACS) Analysis of hCAR and hCEA Expression

Chinese hamster ovary (CHO) and CHO-CAR ceils were evaluated for hCAR expression using anti-CAR mouse mAb (Millipore, Billerica, Mass.) and an anti-mouse ALEXAFLUOR® 488-labeled goat IgG (Molecular Probes, Eugene, Oreg.). For evaluation of hCEA expression, cells were stained with anti-human CEA rabbit IgG (Millipore) an anti-rabbit fluorescein isothiocyanate (FITC)-labeled goat IgG (Millipore). Cells were incubated with antibodies for one hour at 4° C. Following incubation with secondary antibodies, the cells were collected, washed three times in FACS buffer and approximately 10,000 cells were illuminated at 488 nm and fluorescence was detected in the FITC (525/20 nm) channel Non-specific fluorescence was detected using a 575/30 nm emission filter in the PI channel.

### Statistical Analysis

All error terms are expressed as the standard deviation of the mean. Significance levels for comparison of differences between groups in the experiments were analyzed by Student's f test. All reported p-values are two-sided. The differences were considered significant when p-value was <0.05.

### EXAMPLES

The present teachings include descriptions provided in the examples that are not intended to limit the scope of any aspect, or claim. Unless specifically presented in the past tense, an example can be a prophetic or an actual example. The following non-limiting examples are provided to further illustrate the present teachings. Those of skill in the art, in light of the present disclosure, will appreciate that many changes can be made in the specific embodiments that are disclosed and still obtain a like or similar result without departing from the spirit and scope of the present teachings.

### Example 1

This example illustrates isolation of the anti-hCEA VHH.

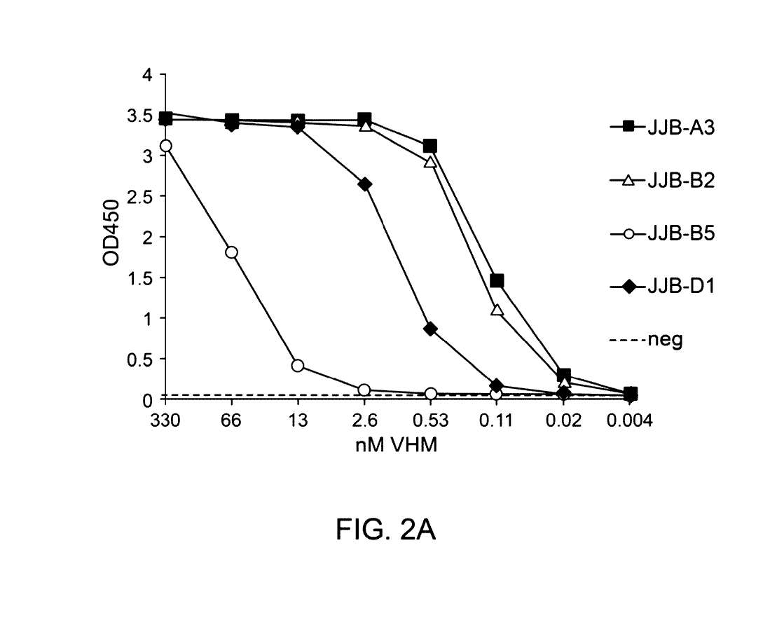

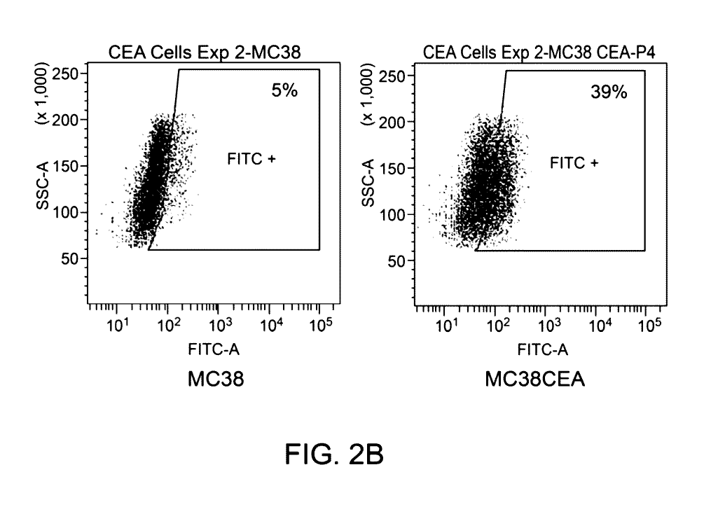

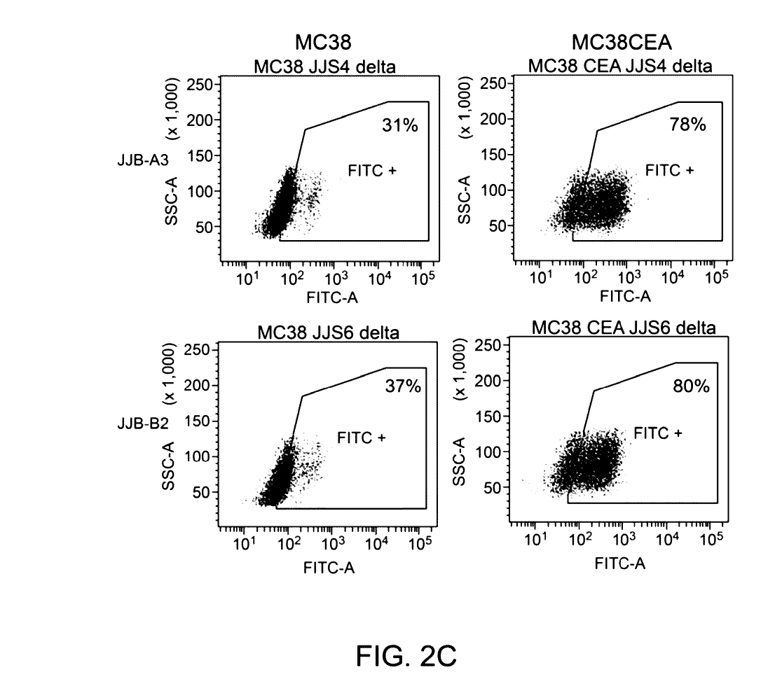

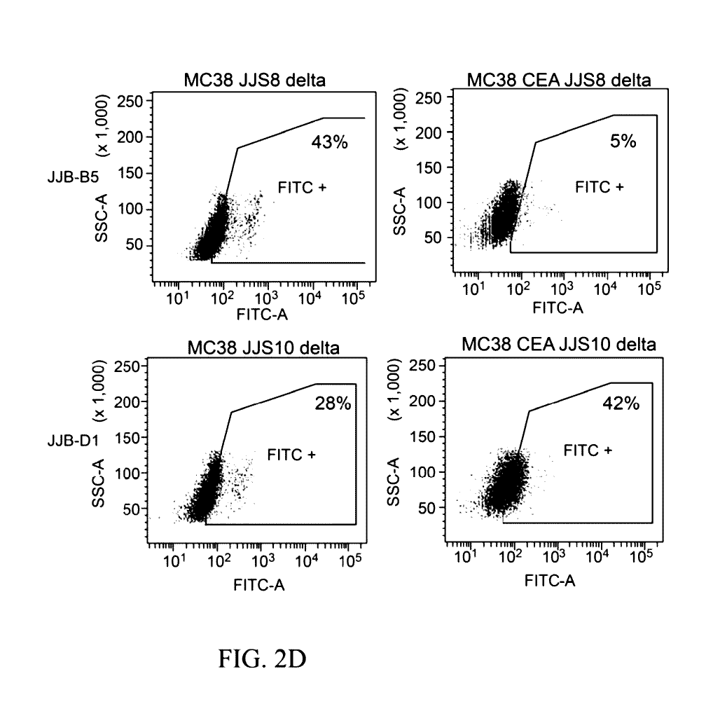

The inventors produced a VHH-display library from peripheral blood lymphocytes RNA of alpacas at the peak of immune response to the hCEA antigen, A VHH phage display library was prepared representing the VHH repertoire from two alpacas immunized, with purified hCEA protein and screened to identify VHHs that bind to hCEA. Four VHHs (JJB-A4, JJB-B2, JJB-B5, JJB-D1) representing apparently unrelated hCEA-binding VHH groups were selected and characterized for hCEA affinity by dilution ELISA (FIG. 2A). The plates for enzyme-linked immunosorbent assay were coated with purified hCEA protein and then purified VHH were added in wells at various concentrations. (FIG. 2A) Bound VHHs were detected with HRP/anti-E-tag mAb. Each point represents a mean of six readings obtained in two separate experiments. The EC₅₀ values are the VHH concentration that produced 50% maximum signal on the ELISAs. These results indicated that JJB-A4, JJB-B2 and JJB-D1bound hCEA with EC₅₀ of approximately 0.15, 0.2 and 1 nM, respectively, while JJB-B5 had lower affinity for hCEA (EC₅₀-50 nM). The four VHHs were also characterized by FACS for their ability to recognize hCEA expressed on the surface of mammalian cells. For this study hCEA expression in MC38 (hCEA−) and MC38CEA (hCEA+) murine colon cancer cells were evaluated by FACS analysis. MC38CEA (hCEA+) and MC38 (hCEA−) mouse colon cancer cells were stained with anti-human CEA rabbit IgG and an anti-rabbit FITC-labeled goat IgG and subjected to FACS analysis. The levels of hCEA expression varied in different cell lines, MC38CEA cells demonstrated higher number of hCEA expressing cells in comparison with MC38 cells (FIG. 2B). As shown in FIG. 2C-2D, JJB-A3 and JJB-B2 both recognized cells expressing hCEA. Bound anti-CEA VHHs were detected using anti-E-tag FITC-conjugated goat Ab using FASC analysis. MC38CEA and MC38 cells incubated with 100 ng/ml of JJB-A3, JJB-B2, JJB-B5, and JJB-D1 VHHs. There was an increased number of CEA+ cells which bound JJB-A3 and JJB-B2 VHHs (78% and 80%, respectively), and JJB-B2 was selected for further studies.

### Example 2

This example illustrates recombinant Ad vectors.

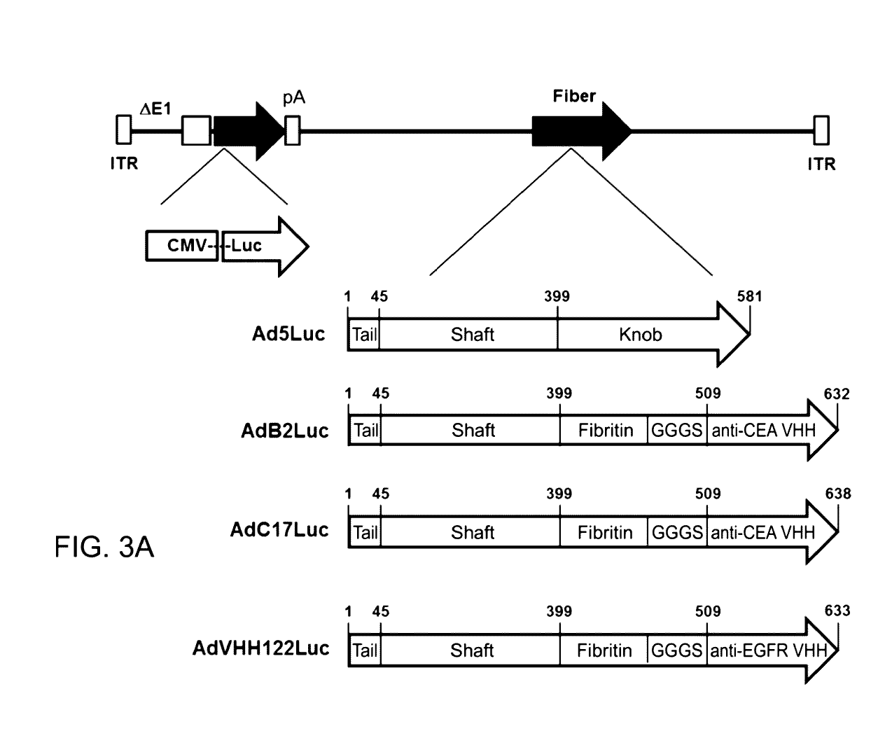

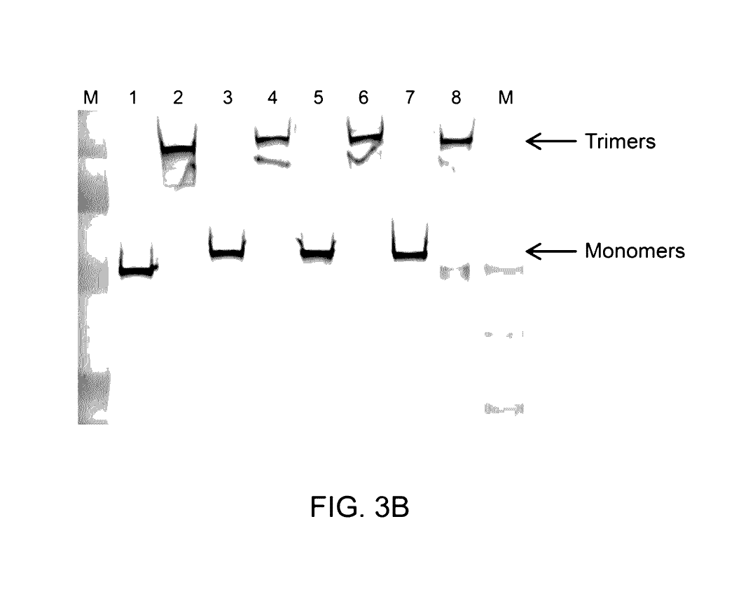

For this study the inventors developed a panel of recombinant Ad5-based vectors expressing the firefly luciferase (Luc) gene under transcriptional control of the human cytomegalovirus (CMV) major immediate-early enhancer/promoter element (FIG. 3A). The chimeric fiber-fibritin (FF) protein containing the N-terminal Ad5 fiber tail region fused to the entire fibritin protein with the trimerizing foldon domain of bacteriophage T4 following by Gly-Gly-Gly-Gly-Ser (SEQ ID NO:5) peptide linker connected to the VHH ORF. AdVHH122Luc and Ad5Luc vectors expressing anti-epidermal growth factor receptor (EGFR) FF-VHH chimera and wild-type Ad5 fiber and the CMV-Luc cassette, respectively, were used as isogenic control Ad vectors.

To demonstrate the incorporation of the targeting FF-VHH fusion proteins into the virus, 5×10⁹ v.p. of boiled and unboiled purified Ads were loaded in each lane and subjected to SDS-PAGE followed by Western blot analysis using anti-fiber mAb. (Fiber protein expression was detected using anti-fiber mAb (clone 4D2)). As shown in FIG. 3B, genetic incorporation of VHHs produced stable fusion with fiber-fibritin molecules that maintained trimerization potential of chimeric proteins. Equal amounts (5×10⁹ v.p.) of purified the FF-VHH modified Ad vectors including AdC171Luc (lanes 3 and 4), AdB2Luc (lanes 5 and 6), and AdVHH122Luc (lanes 7 and 8) or the fiber unmodified control Ad5Luc vector (lanes 1 and 2) were loaded in each lane with boiling in a sample buffer (lanes 1, 3, 5 and 7) or without boiling (lanes 2, 4, 6 and 8) and separated on SDS-PAGE followed by transfer to a PVDF membrane.

### Example 3

This example illustrates binding properties of the Ad vectors to the hCEA.

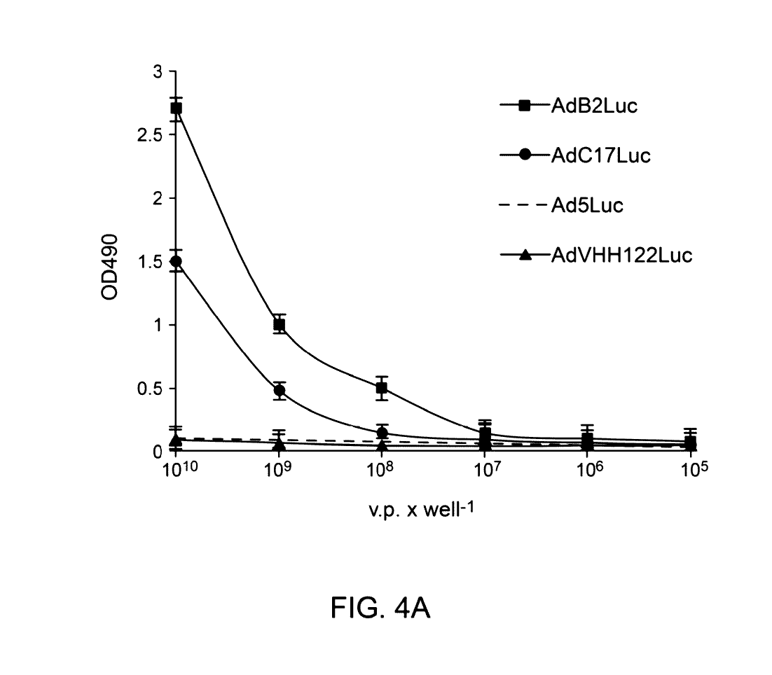

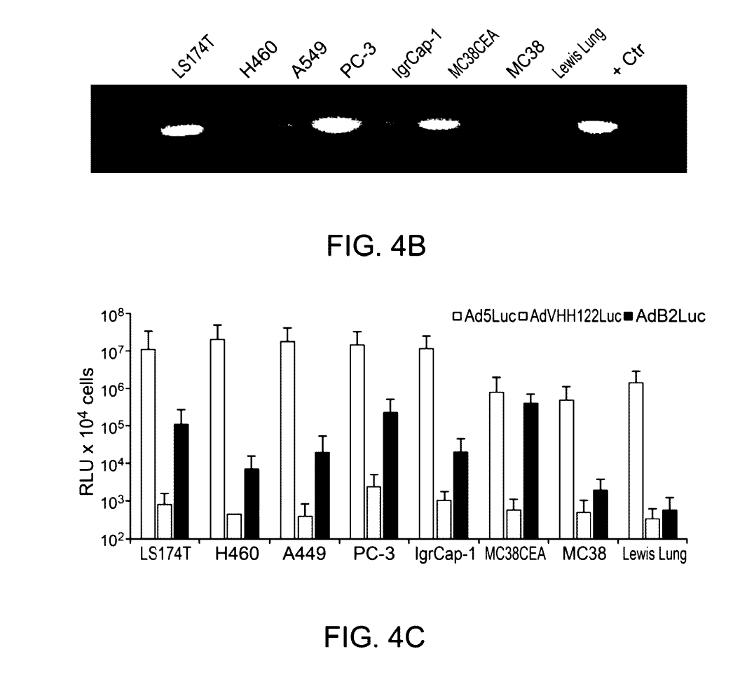

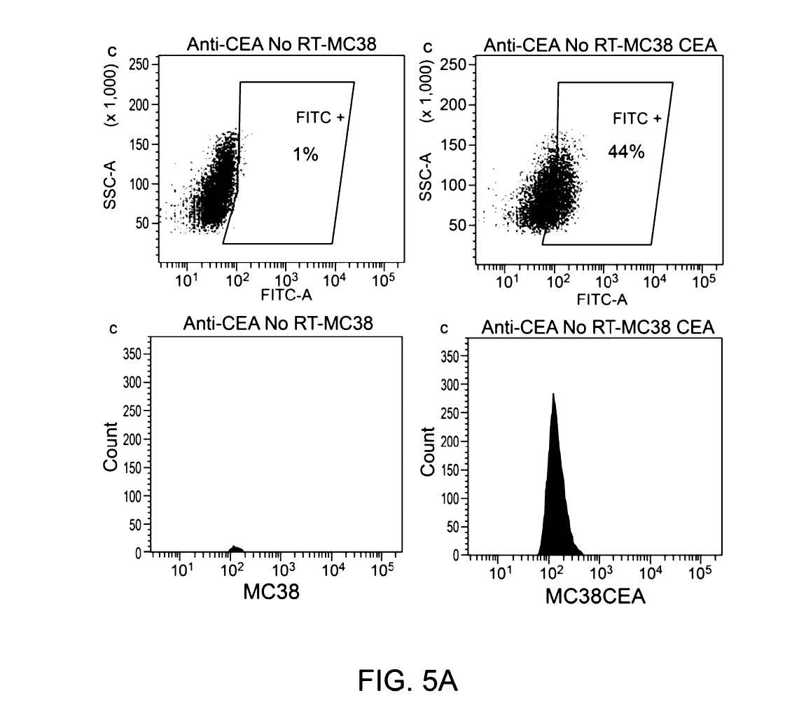

To evaluate specificity of binding recombinant Ad vectors to hCEA, a purified AdB2Luc and AdC17Luc vectors displaying anti-hCEA FF-VHH chimera, AdVHH122Luc expressing anti-EGFR FF-VHH fusion and Ad5Luc with wild-type fiber protein were incubated with the hCEA protein adsorbed on surface 96-well plate (FIG. 4A). The plates for enzyme-linked immunosorbent assay were coated with purified hCEA protein and then purified Ad virions were added in wells at various concentrations. Bound viral particles were detected by using polyclonal anti-adenovirus goat Ab. Each point represents a mean of six readings obtained in two separate experiments, with the error bars showing standard deviations (s.d.). Results of ELISA using anti-fiber Ab revealed a significant degree of binding of AdB2Luc and AdC17Luc-expressing anti-hCEA FF-VHH to the hCEA in contrast to AdVHH122Luc and Ad5Luc which demonstrated no binding to the hCEA.

For initial evaluation of transduction efficiency and specificity of targeting of Ad5 vector containing anti-hCEA VHH, several cancer cell lines were infected with AdB2Luc displaying anti-hCEA FF-VHH chimera, AdVHH122Luc with anti-EGFR FF-VHH fusion and wild-type Ad5Luc. The inventors determined endogenous hCEA mRNA expression in cells using RT-PCR. Total RNA was extracted from human and mouse cancer cells, the first-strand cDNA was synthesized using random hexamer primers and used as the template for PGR. Products of PGR were analyzed by 1% agarose electrophoresis with ethidium bromide staining. As shown in FIG. 4B, LS174T, PC-3, and MC38CEA cells demonstrated high levels of hCEA mRNA expression in comparison with other tested cells, whereas MC38 and Lewis Lung cells showed the lowest levels of hCEA mRNA expression. Since all tested Ad vectors comprise identical CMV promoter Luc gene cassettes. Ad transduction was compared by evaluation of Luc expression in the infected cells. Human and mouse cancer cells were infected with 5×10³ v.p. per cell of Ads. Forty-eight hours after infection, cells were harvested and Luc expression was analyzed. Levels of Luc expression were varied in different cell lines in proportion to viral doses of infection (results not shown). As illustrated in FIG. 4C, infection with AdB2Luc yielded lower Luc expression compared to Ad5Luc, with the exception of MC38CEA mouse colon cancer cells, and relative levels of Luc expression of cancer cells were correlated with levels of hCEA mRNA expression. Data are presented as relative light units (RLU) per 1×10⁴ cells and bars represent the mean±s.d.

### Example 4

This example illustrates specificity of Ad mediated gene transfer.

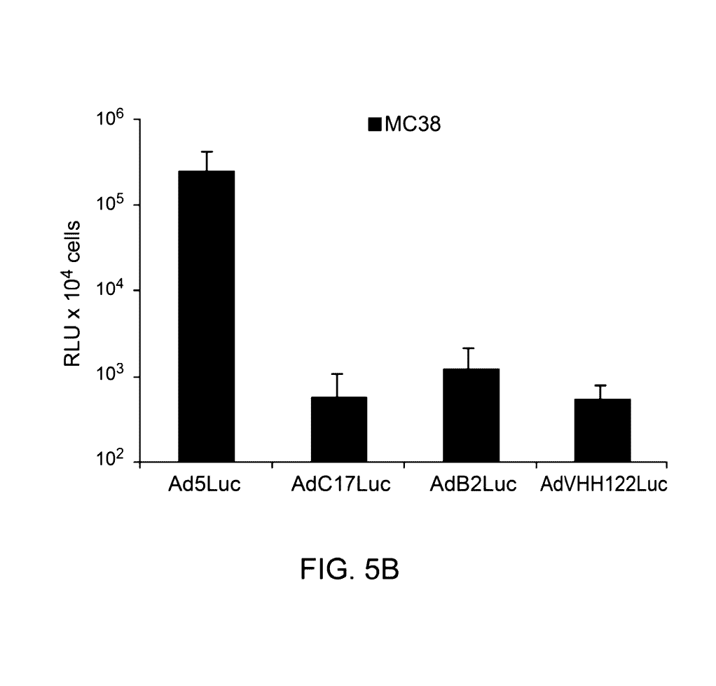

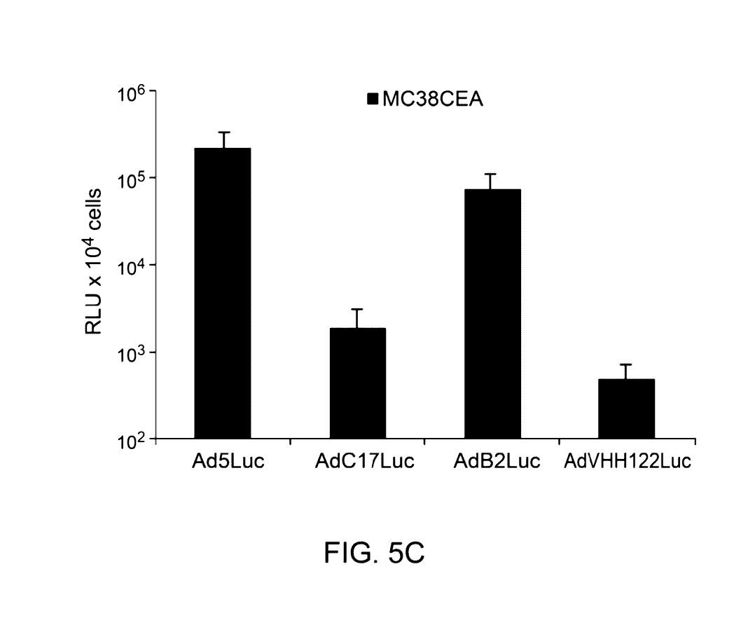

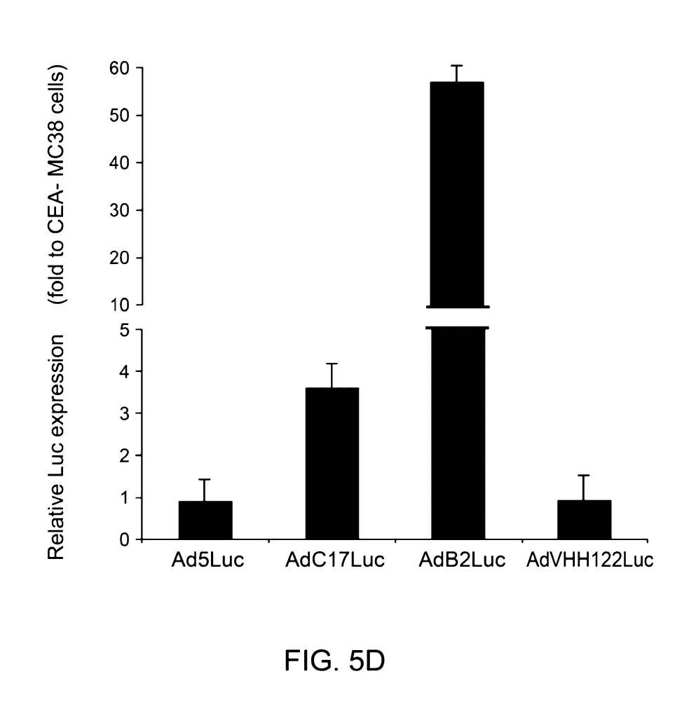

The inventors investigated whether AdB2Luc and AdC 17Luc vectors encoding different anti-hCEA FF-VHHs retain specificity for the appropriate CEA expressing (CEA+) cells. For this study hCEA expression on surface of MC38 (hCEA−) and MC38CEA (hCEA+) murine colon cancer cells were evaluated by FACS analysis. MC38 (hCEA−) and MC38CEA (hCEA+) mouse colon cancer cells stained with anti-human CEA rabbit IgG and an anti-rabbit FITC-labeled goat IgG and subjected to FACS analysis. As shown in FIG. 5A, levels of hCEA expression varied in different cell lines. MC38CEA cells demonstrated higher number of hCEA expressing cells (44 %), in comparison with MC38 cells (1%). To evaluate specificity of Ad mediated gene transfer, MC38 (FIG. 5B) and MC38CEA (FIG. 5C) cells were infected with 5×10³ v.p. per cell of AdB2Luc, AdC17Luc, AdVHH122Luc and Ad5Luc and level of Luc reporter gene expression was detected at 48 hours after infection. As shown in FIG. 5D, infection with AdB2Luc produced more than 55-fold increase (P<0.05) of reporter gene expression in hCBA-positive MC38CEA cells in comparison MC38 cells. In contrast, Luc expression was only slightly (˜4-fold) increased in hCEA+ cells following AdC17Luc infection. The relative Luc expression was increased in AdB2Luc infected MC38CEA cells in comparison with MC38 cells (*, P<0.05 vs MC38). There were no significant differences across Luc expression in tested cells infected with AdVHH122Luc and Ad5Luc. Data are presented as relative light units (RLU) per 1×10⁴ cells and bars represent the mean±s.d.

### Example 5

This example illustrates CAR-independent AdB2Luc infection.

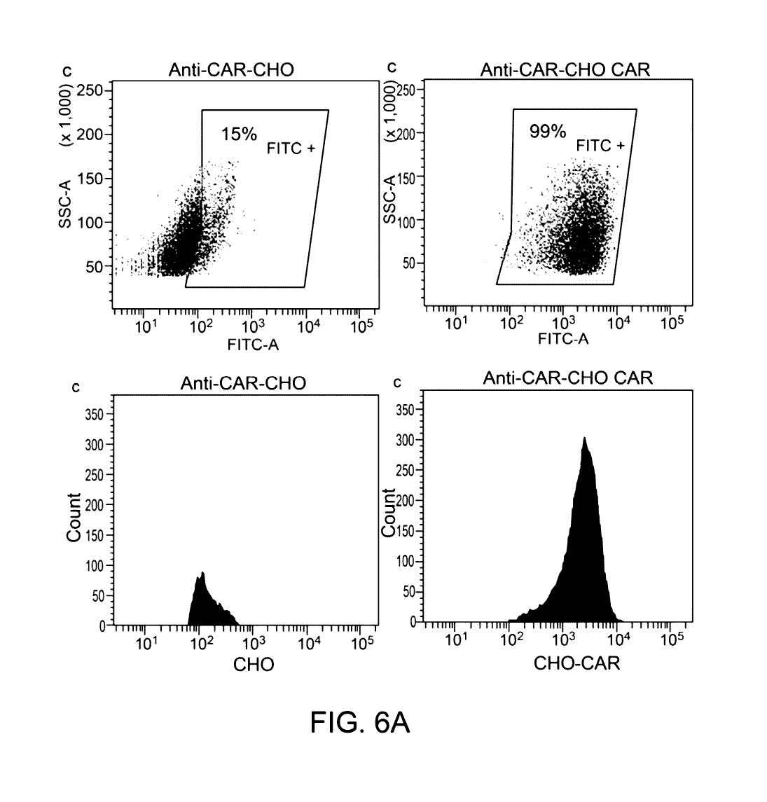

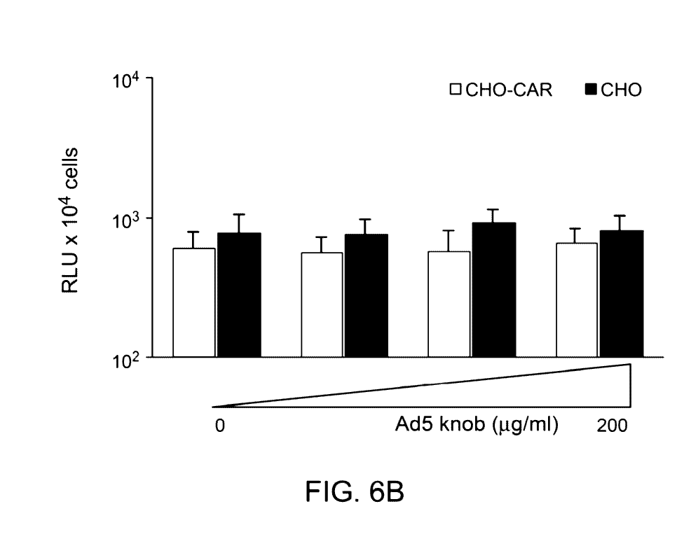

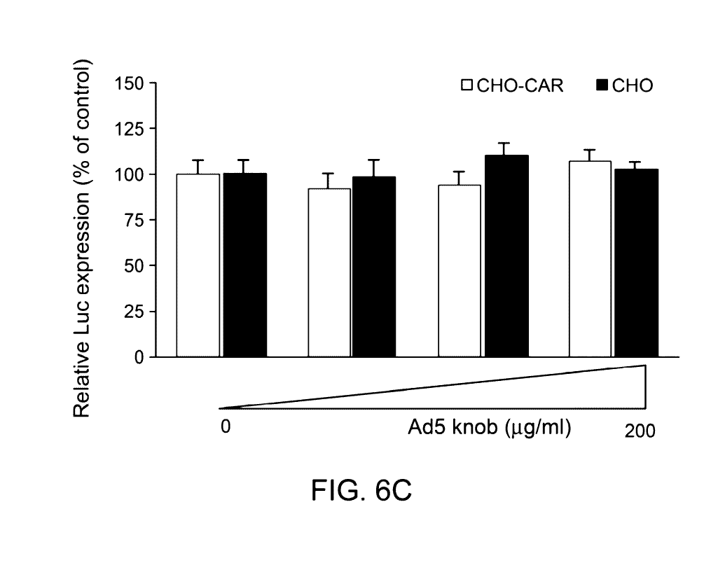

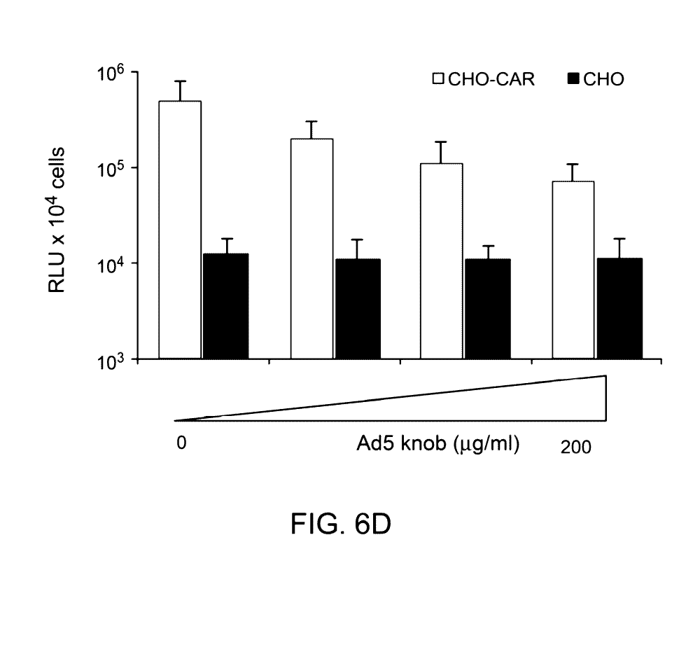

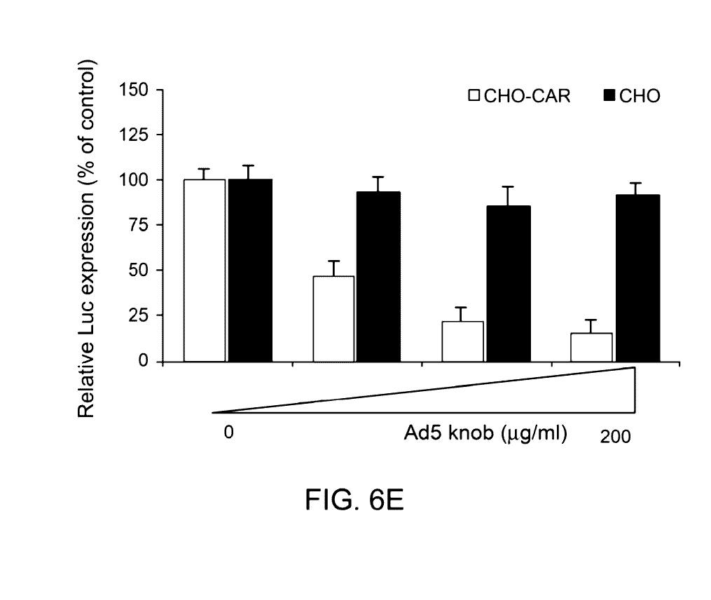

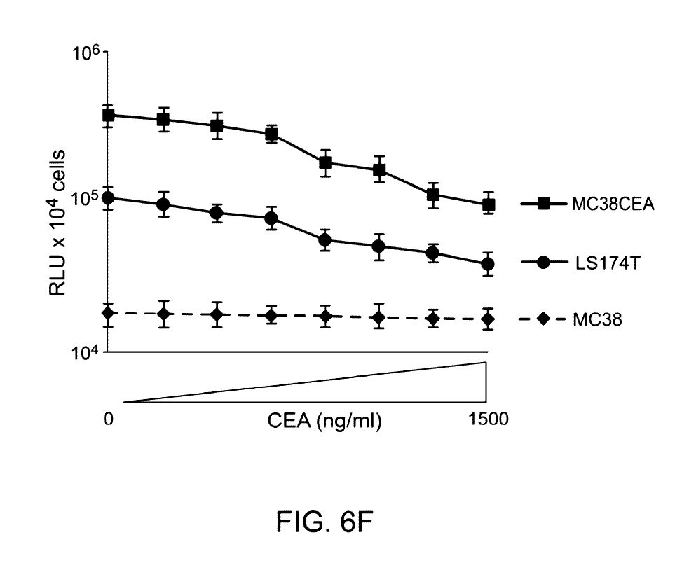

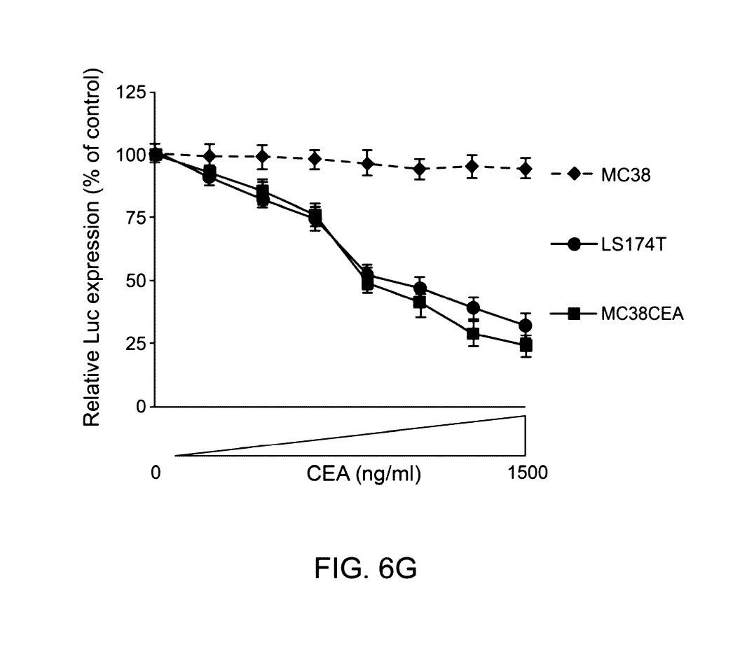

The inventors evaluated whether modification in the Ad5 fiber resulted in ability of AdB2Luc to CAR-independent binding and infection in vitro. The inventors expressed recombinant Ad5 knob and evaluated the purified proteins in Western blotting using anti-His mAb (data not shown). The inventors evaluated the hCAR expression in hCAR-expressing CHO-CAR (Santis, G., et al., J. Gen. Virol., 80, 1519-27, 1999) and CHO Chinese hamster ovary cells by staining with anti-human CAR rabbit IgG and an anti-rabbit FITC-labeled goat IgG and using then FACS analysis. As shown in FIG. 6A, CHO-CAR cells demonstrated a high level of hCAR expression (99%) in comparison with CHO cells (15%). To Investigate whether AdB2Luc vector encoding anti-hCEA FF-VHH produces CAR-independent infection hCAR-expressing CHO-CAR and hCAR-negative CHO cells were pretreated with different concentrations of recombinant Ad5 knob or BSA at one hour prior to infection with AdB2Luc or Ad5Luc. Luciferase activity was detected in the lysates of infected cells at 48 hours postinfection. Data are presented as RLU per 1×10⁴ cells and bars represent the mean±s.d.

As shown in FIG. 6B and FIG. 6C, preincubation with Ad5 knob did not block AdB2Luc-mediated Luc gene expression in CHO-CAR cells. (Luciferase activity is given as percentages of the activity in presence of Ad5 knob compared with mock-treated cells (FIG. 6C)). In contrast, Ad5Luc infection was efficiently blocked by recombinant Ad5 knob protein in a dose-dependent manner (FIG. 6D). CHO (hCAR−) and CHO-CAR (hCAR+) cells were preincubated with soluble Ad5 knob protein at different concentration and infected with 5×10³ v.p. per cell of Ad5Luc. Data are presented as RLU per 1×10⁴ cells and bars represent the mean±s.d. (FIG. 6D). Incubation CAR expressing cells with 200 mg/ml of Ad5 knob resulted in ˜85% decreased Luc expression following infection with Ad5Luc. Luciferase activity is given as percentages of the activity in presence of Ad5 knob compared with mock-treated cells (FIG. 6E). There was no blocking effect of incubation of CHO cells with recombinant Ad5 knob for both Ad5Luc- and AdB2Luc-mediated gene transfer in the same experiment.

### Example 6

This example illustrates dose-depended inhibition, of AdB2Luc gene transfer by hCEA.

To confirm a specificity of anti-hCEA FF-VHH mediated AdB2Luc infection the inventors evaluated hCEA-mediated inhibition of Luc gene transfer. Human colon cancer LS174T cells were used, as a positive control for hCEA expression (Shi, Z. R., et al., Cancer Research, 43, 4045-4049, 1983). AdB2Luc was preincubated with different concentration of hCEA or BSA for one hour before infection of MC38 and MC38CEA mouse colon cancer cells and LS174T cells. (MC38 and MC38CEA mouse colon cancer cells and LS174T human colon cancer cells were infected with AdB2Luc at 5×10³ v.p. per cell). Forty-eight hours after infection cancer cells were lysed and Luc activity was measured Data are presented as RLU per 1×10⁴ cells and bars represent the mean±s.d. (FIG. 6F).

Results of gene transfer blocking assay demonstrated a dose-depended inhibition of Luc gene transfer in both CEA+ cell lines following pretreatment of AdB2Luc with hCEA. Gene transfer efficiency of AdB2Luc was significantly reduced after incubation with blocking protein, and only 24% and 30% of Luc expression was retained following infection of MC38CEA and LS174T cells, respectively, after incubation with 1500 ng/ml of hCEA (FIG. 6G). In contrast, preincubation of AdB2Luc with hCEA protein at the highest concentration did not affect in Ad-mediated gene transfer in the hCEA negative MC38 cells, Luciferase activity is given as percentages of the activity in comparison with BSA-treated Ad.

### Example 7

This example illustrates radiation-inducible increasing of AdB2Luc infection.

High energy x-rays are tissue penetrating, cytotoxic, and can be tumor targeted to a focal point. Cells respond to ionizing radiation with the activation of specific early and later response genes. Preclinical studies have shown the up-regulation of CEA mRNA and protein expression in clinical tumor samples as well as human cancer cell lines following irradiation (Hareyama, M., et al., Cancer 67, 2269-2274, 1991; Garnett, C. T., et al. Cancer Research 64, 7985-7994, 2004; and Matsumoto, H., et al. Anticancer Research, 19, 307-311, 1999). The inventors hypothesized that the radiation-inducible CEA overexpression could be used to regulate Ad mediated transgene expression in irradiated tumor cells. The inventors sought to determine whether ionizing radiation alters AdB2Luc transduction.

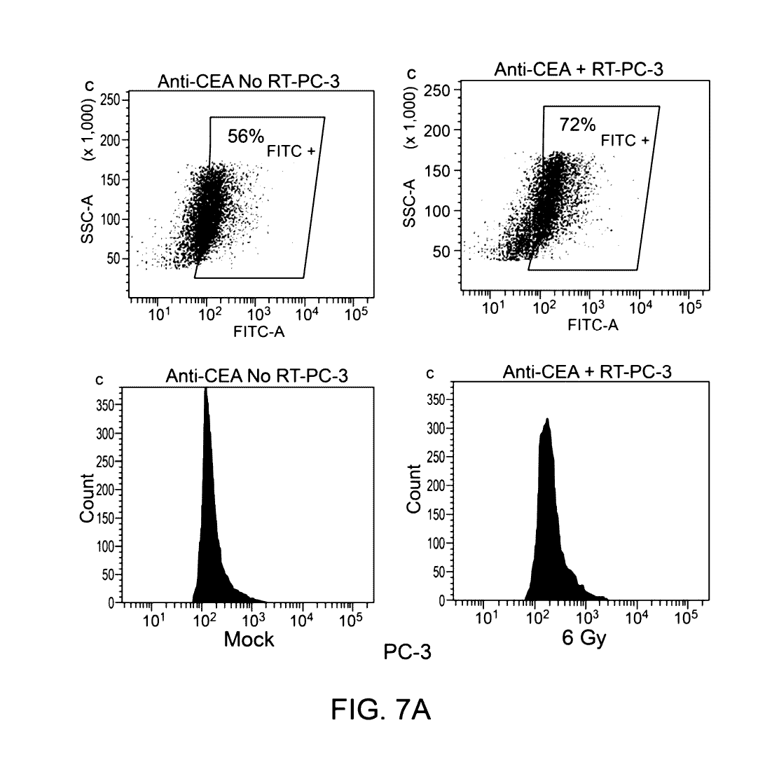

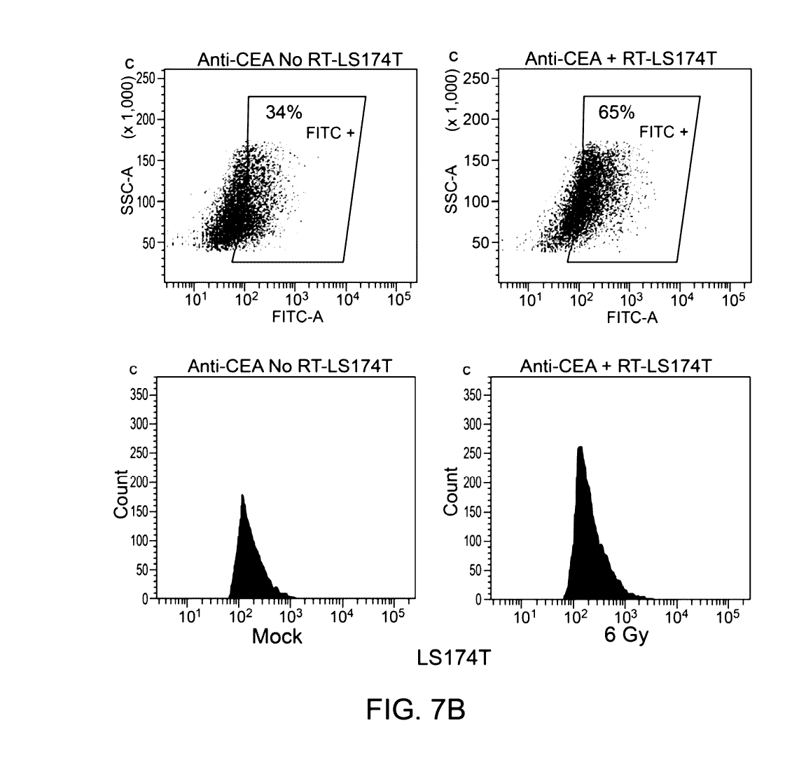

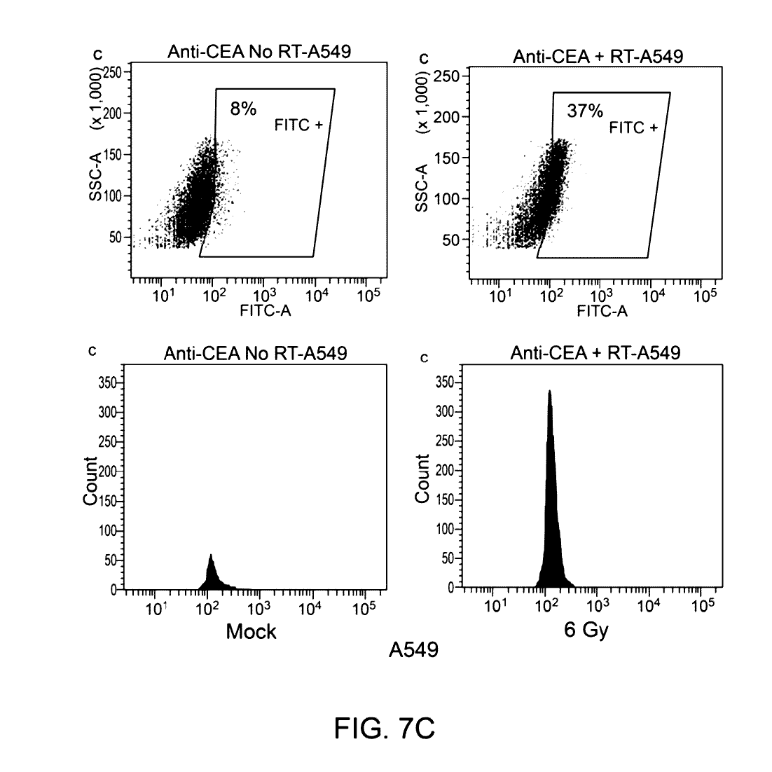

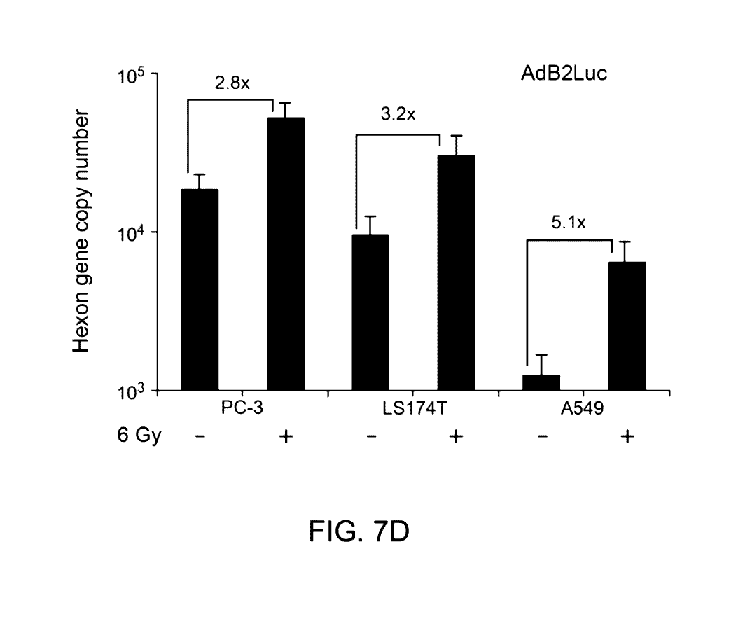

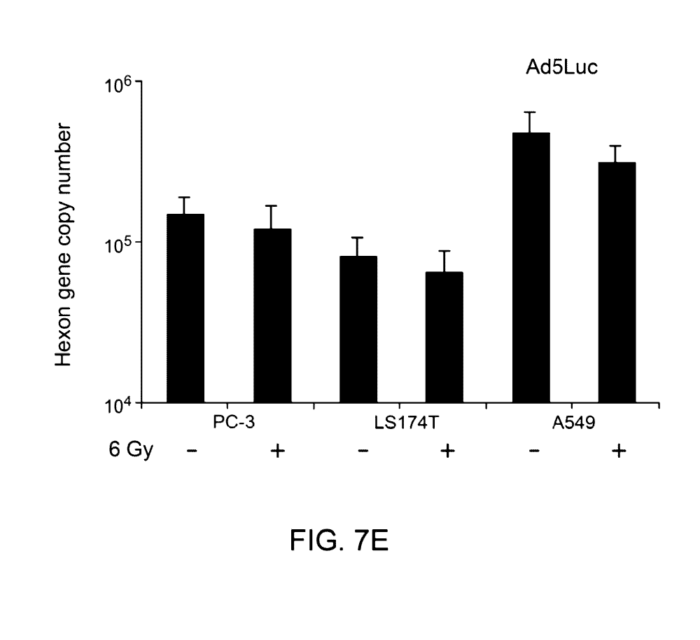

The inventors evaluated the hCEA expression following radiation treatment of cancer cells. For evaluation of hCEA expression, PC-3 (FIG. 7A), LS174T (FIG. 7B) and A549 (FIG. 7C) cells were mock-irradiated or irradiated at 6 Gy, stained with anti-human CEA Ab and subjected to FACS analysis. Human cancer cells demonstrated high (PC-3, FIG. 7A), mediate (LS174T, FIG. 7B) and low (A549, FIG. 7C) basal levels of hCEA expression were mock-treated or irradiated at 6 Gy and then the hCEA expression was evaluated using FACS. There was a time-dependent and transient increase of hCEA expression in all tested cells which was reached a peak of number CEA+ cells at 24 hours following irradiation at 6 Gy and slow declined to the basal levels of expression at 72 hours post treatment (data not shown). As shown in FIG. 7A-C, the number of hCEA expressed/FITC+ cells was increased by 1.3-fold in PC-3 prostate cancer cells, 1.9-fold in LS174T colorectal adenocarcinoma cells and 4.6-fold in A549 lung cancer cells at 24 hours post radiation treatment

Twenty-four hours after radiation treatment at 6 Gy human cancer ceils were infected with 5×10³ v.p. per cell of AdB2Luc (FIG. 7D) or Ad5Luc (E) recombinant vectors. After incubation for one hour, total DNA was extracted and quantitative analysis of the Ad hexon gene expression was performed using TAQMAN® PCR. Data are means of hexon copy numbers per 1 ng of total DNA±s.d. As illustrated in FIG. 7D, the copy number of Ad hexon gene was increased by 2.8-fold in PC-3 cells, 3.2-fold in LS174T cells and 5.1-fold in A549 cells in comparison with mock-treated cells and relative levels of AdB2Luc transduction were correlated with levels of increased hCEA expression following exposure of cancer cells to ionizing irradiation. In contrast, the Ad hexon gene copy number was slightly decreased in irradiated cells following Ad5Luc infection (FIG. 7E).

Taken together, obtained data demonstrates that AdB2Luc vector with genetically incorporated anti-hCEA VHH into a de-knobbed Ad5 fiber-fibritin chimera retains hCEA recognition functionality and provides specificity of gene transfer of capsid-modified AdB2Luc vector in vitro.

### Example 8

This example illustrates the expression of conditionally implicative hCEA-targeted fiber-fibritin-sdAb protein.

The fiber-fibritin-hCEA protein was created as described previously (Kaliberov, SA 2014 Lab Invest 94: 893-905). Briefly, alpacas were immunized with soluble human CEA (ProNique Scientific, Castle Rock, Colo.) and sdAbs against hCEA were acquired by phage biopanning. Of all screened sdAb clones B2 was the most efficient in binding hCEA. From these results, the inventors produced a panel of Ad5 based vectors expressing the E1a gene under transcriptional control of the CXCR4 promoter element including Ad.CXCR4E1 with wild-type Ad5 fiber, Ad.CXCR4E1.B2 vector with a fiber-fibritin chimera expressing antihCEA sdAb (clone B2), as well as replication-deficient recombinant adenoviruses, Ad.CXCR4Luc and Ad.CMVLuc encoding the firefly luciferase (Luc) gene under control of the CXCR4 or human cytomegalovirus (CMV) promoter (created as described in (Kaliberov, S.A., Lab. Invest., 94, 893-905, 2014), respectively. To create Ad.CXCR4E1.B2, B2 was fused in single open reading frame with a chimeric fiber-fibritin protein which contained the N-terminal Ad5 fiber tail region fused to the trimerizing domain of the fibritin protein of bacteriophage T4 followed by a peptide linker (G-G-G-S) connected to the B2 sdAb as described previously (Noureddini, et al., Virus Res., 116, 185-195, 2006). The fiber-fibritin-B2 (FFB2) protein was retrieved from pKan566FFB2 using EcoR1 and Sal I restriction sites. Recombinant adenovirus genomes were generated by homologous DNA recombination in E. coli BJ5183 between the restricted FFB2 and Ad5 fiber gene deleted pVK500C.CXCR4E1, resulting in pVK500C,CXCR4E1,B2. Insertion of the fiber gene was confirmed by PCR and partial sequence analysis. The plasmid was linearized using Pac I restriction and transfected into 293F28 cells using SuperFect™ Transfection Reagent (Qiagen, Chatsworth, Calif.). 293F28 cells stably express the native Ad5 fiber; therefore, a mixture of fibers was present on the viruses rescued at this point. After an additional round of amplification in 293F28 cells, viruses were amplified in Ad5-fiber negative HEK293 cells to obtain viral particles containing only the B2-fiber. Viruses were propagated in HEK293 cells and purified twice by CsCl gradient: centrifugation. Viral particles were dialyzed against 10% glycerol in phosphate-buffered saline (PBS). Viral particles (vp) were quantified by measuring absorbance of the dissociated virus at A₂₆₀ nm using a conversion factor of 1.1×10¹² vp per absorbance unit.

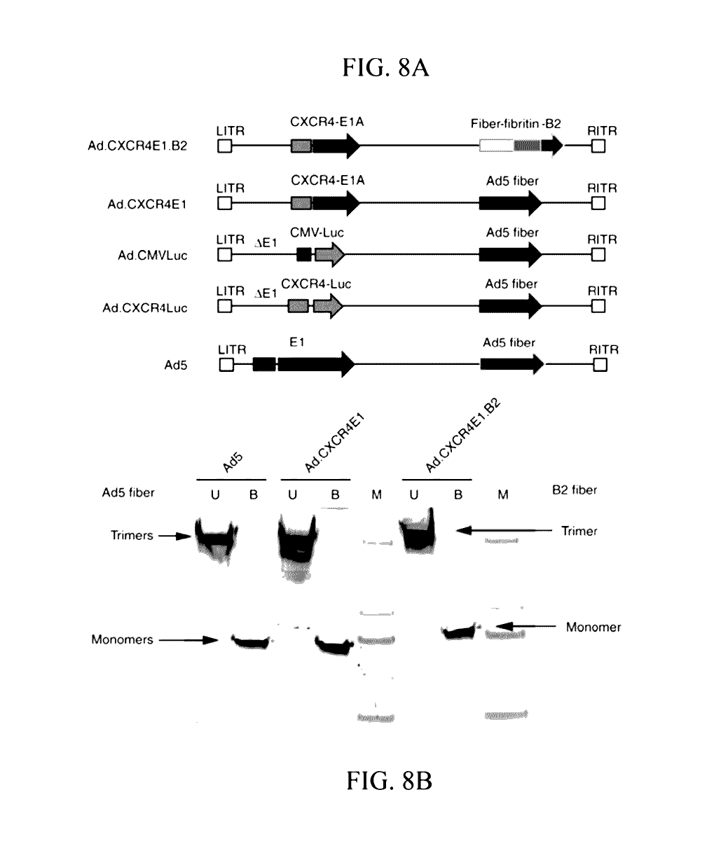

The Ad.CXCR4E1. conditionally replicative vector and replication deficient Ad.CMVLuc and Ad.CXCR4Luc vectors were created as described before. Wild-type Ad5 was kindly provided by Dr H Ugai (Washington University in St Louis, St Louis, Mo.) for use as a control virus. A schematic overview of the vectors used in this study is presented in FIG. 8A.

To confirm the incorporation of the chimeric fiber-fibritin-sdAb protein into Ad.CXCR4E1B2, boiled and unboiled purified adenovirus vectors were analyzed by western blotting using an anti fiber mAb. Samples containing 5×10⁹ viral particles were preincubated in Laemmli sample buffer for 10 minutes at 99° C. or 25° C. for seminative conditions. Proteins were separated using a 4-20% gradient polyacrylamide Precise™ Protein gel (Thermo Scientific, Wilmington, Del.). The proteins were blotted onto polyvinylidene difluoride (PVDF) membranes and developed with the Sigma FAST 3,3′-diammobenzidine system (Sigma-Aldrich, St Louis, Mo.) according to the manufacturer's protocol Anti-Ad5 fiber mAb (4D2, Thermo Scientific) and goat-anti-mouse Ig-HRP (DakoCytomation Denmark A/S, Glostrup, Denmark) were used for Ad5 fiber protein detection. Equal amounts (5×10⁹ vp) of purified viral particles from Ad5, Ad.CXCR4E1 and Ad.CXCR4E1.B2 were loaded in sample buffer in each lane without (lane 1, 3, and 6) or with boiling (lane 2, 4, and 7). Proteins were separated on a SDS-PAGE gel followed by western blot transfer to a PVDF membrane. Fiber protein expression was detected using antifiber mAb. Predicted molecular weight (MW) of wild-type Ad5 fiber monomers is 61.6 kDa and MW 67.7 kDa for fiber-fibritin-sdAb. One representative of three different experiments is shown in FIG. 8B. FIG. labels are as follows; B, boiled; LITR, left inverted terminal repeat: M, marker; PVDF, polyvinylidene difluoride; RITR, right inverted terminal repeat; U, unboiled; ΔE1, E1 deleted. As expected, the chimeric fiber-fibritin-sdAb in Ad.CXCR4E1,B2 is slightly larger (with predicted molecular weight 67.7 kDa for fiber-fibritin-sdAb monomer) than the native Ad5 (molecular weight of wild-type Ad5 fiber protein is 61.6 kDa) and fiber displayed in Ad.CXCR4E1 and Ad5. Genetic incorporation of sdAbs produced a stable fusion with fiber-fibritin molecules that maintained the trimerization potential of chimeric fiber-fibritin-sdAb proteins under native conditions (FIG. 8B).

### Example 9

This example illustrates that Ad.CXCR4E1.B2 demonstrates hCEA-selective binding.

To evaluate specificity of Ad.CXCR4E1B2 transduction, MC38 and MC38CEA murine colon adenocarcinoma cells were used. To determine the levels of hCEA surface expression, approximately 1×10⁶ cells were collected, washed with PBS, and stained with anti-hCEA rabbit IgG (Millipore, Billerica, Mass.) and antirabbit FITC-labeled goat IgG (Millipore) for one hour at 4° C. Levels of hCAR surface expression were measured with anti-hCAR mAb (RemB), kindly provided by Dr. J. Douglas (University of Alabama at Birmingham) and antimouse FITC-labeled goat IgG (Molecular Probes, Eugene, Oreg.). Mouse IgG1 negative control (Millipore) and rabbit IgG isotype control (Thermo Scientific, Rockford, Ill.) were used as isotype controls. After washing in PBS for three times, cells were resuspended in FACS buffer. Approximately 1×10⁴ cells were illuminated at 488 nm, detecting fluorescence in the FITC (525/20 nm) channel.

As expected, FACS analysis showed no hCAR expression in both cell lines and no hCEA expression in the MC38 cells (Table 1) in contrast to the high levels of hCEA expression in MC38CEA cells (Table 1),

TABLE 1
Flow cytometry analysis of hCEA and hCAR surface expression

% of positive cells
(mean of fluorescence intensity)^{a}

| Cell line | hCEA | | hCAR | |
|---|---|---|---|---|
| MC38 | 1 + 3 | (4 + 1) | 8 + 7 | (2 + 2) |
| MC38CEA | 95 + 8 | (48 + 19) | 4 + 3 | (2 + 1) |
| LS174T | 67 + 14 | (12 + 9) | 61 + 17 | (28 + 16) |
| HS766T | 35 + 17 | (9 + 8) | 2 + 1 | (3 + 3) |
| U118MG | 2 + 1 | (2 + 3) | 2 + 3 | (5 + 7) |
| U118-hCAR | 1 + 1 | (1 + 3) | 99 + 5 | (581 + 76) |
| THLE-3 | 10 + 9 | (4 + 3) | 55 + 11 | (44 + 17) |

Both cell lines were infected with Ad.CXCR4E1.B2 or Ad.CXCR4E1 for one hour, washed, total DNA was extracted and subjected to quantitative real-time PCR (qPCR) analysis. Cell binding by Ad.CXCR4E1.B2 was strongly enhanced in the hCEA expressing MC38CEA cells compared to the control vector, while both CRAds had limited binding to the hCEA(−)hCAR(−) MC38 cell line. Cells were seeded 3×10⁵ cells per well in a six-well tissue culture plate and grown overnight. The next day medium was removed, then MC38 and MC38CEA murine colon adenocarcinoma cells were incubated at 37° C. with 1×10³vp per cell of the indicated vector for one hour. Total DNA was isolated from the cells using a QIAAMP® DNA mini Kit (Qiagen, Chatsworth, Calif.).

Ad5 hexon expression was measured using quantitative real-time PCR. Serial tenfold dilutions (from 1×10⁹ to 10 viral particles per reaction) of viral control DNA were included to establish a standard curve. The following primers were used for Ad5 hexon gene detection: Ad5Hexon-fwd (SEQ ID NO:20), Ad5Hexon-rev (SEQ ID NO:21.) and the following TAQMAN® probe was used: Ad5Hexon-probe (SEQ ID NO:22). Mouse β-actin gene expression was used to normalize the samples. The following mouse β-actin probes were used: mβ-actin-fwd: 5′-AGC TGG AGG ACT TCC GAG ACT-3′ (SEQ ID NO:26), mβ-actinrev; 5′-TGG CAC TTC TCC TGC ACC TT-3′ (SEQ ID NO:27), and mβ-actin-probe: 5′-HEX-TAG ACG CCT GCA CAA GCC GCC-BHQ1-3′ (SEQ ID NO:28).

In each reaction, 20 ng of total DNA was added to a total of 10 μl of reaction mixture containing 2× Fast Start TaqMan Probe Master Mix (Roche Applied Science, Indianapolis, Ind.), 333 nmol/l of each primer and fluorogenic probe. Reactions were carried out in triplicates in a 96-well reaction plate (PE Applied Biosystems, Grand Island, N.Y.) in a spectrofluorimetric thermal cycler (LightCycler 480 Real-Time PCR system, Roche Applied Science). The following program was used: denaturation (2 minutes at 95° C.) and amplification with 45 cycles (15 seconds at 90° C. and 60 seconds at 60° C.). The level of binding to MC38 and MC38CEA cells was determined as the Ad hexon gene copy number per 20 ng total DNA.

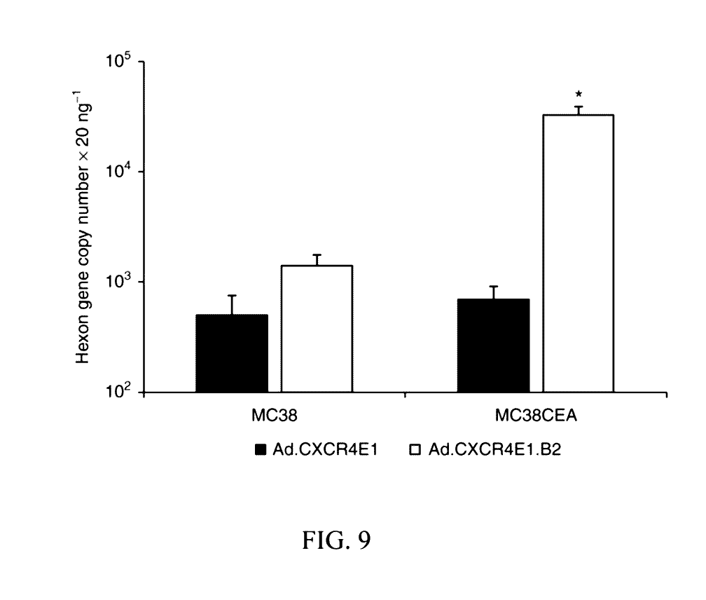

As shown in FIG. 9, Ad.CXCR4E1.B2 binding to hCEA(+) MC38CEA cells was significantly higher (about 25-fold: P<0.01) compared to binding to the hCEA(−) MC38 cells. In contrast, Ad.CXCR4E1 with wild-type Ad5 fiber demonstrated negligible change in binding to MC38CEA cells in comparison with MC38 cells. Also, MC39 cell binding by Ad.CXCR4E1,B2 was slightly higher (about twofold) compared to Ad.CXCR4E1, probably due to structural difference of wild-type Ad5 fiber and fiber-fibritin fusion proteins. Thus, Ad.CXCR4E1,B2 demonstrates hCEA-specific cell binding validating that specificity of the B2 sdAb is maintained in the CRAd context. Data are presented as mean±SD (*P<0.01 versus MC38 cells).

### Example 10

This example illustrates CRAd replication in a human colorectal adenocarcinoma cell line.

To evaluate whether sdAb-targeted CRAds are able to replicate after infection of hCEA(+) cells, a replication assay was performed. Cells were seeded at 3×10⁵ cells per well in six-well tissue culture plates and grown overnight. The next day medium was removed and cells were infected with 1×10³ vp per cell of Ad.CXCR4E1 or Ad.CXCR4E1B2. After incubation at 37° C. for 1 hour, the medium was replaced. Cells were harvested 1, 24, 48, 72, and 120 hours after infection, subjected to three freeze-thaw cycles and centrifuged at 5,000 RPM for 5 minutes. DNA from infected cells was isolated using QIAAMP® DNA Mini Kit (Qiagen, Chatsworth, Calif.). qPCR was performed as described above. Human β-actin gene expression was used to normalize the samples. The following human β-actin primers and probes were used; β-actin-fwd (SEQ ID NO:23), β-actin rev2; 5′TCC ATC TCG CAG TTG GT-3′ (SEQ ID NO:29), and β-actin probe: 5′-HEX-CCC CATCGA GCA CGG CAT CG-BHQ1-3′ (SEQ ID NO:30).

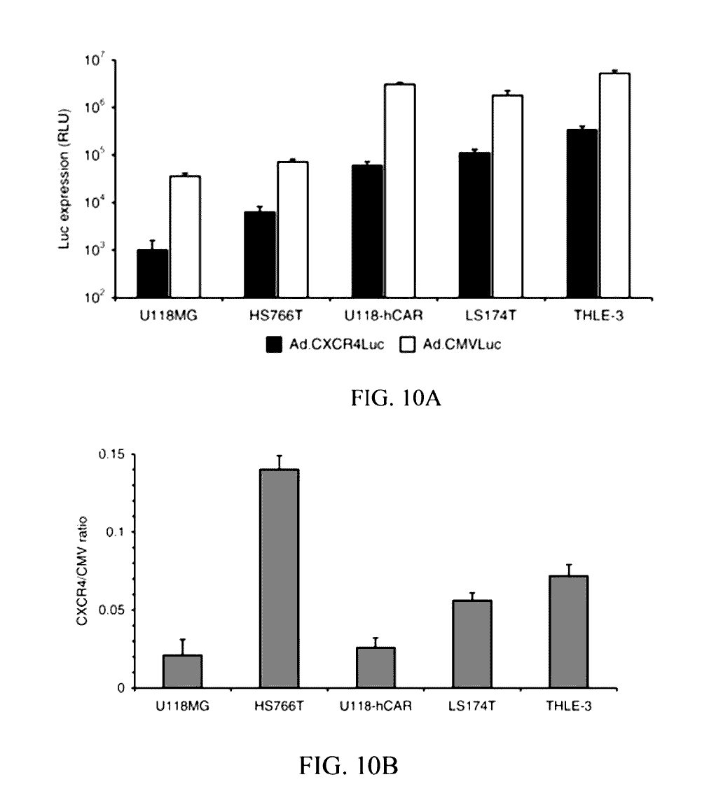

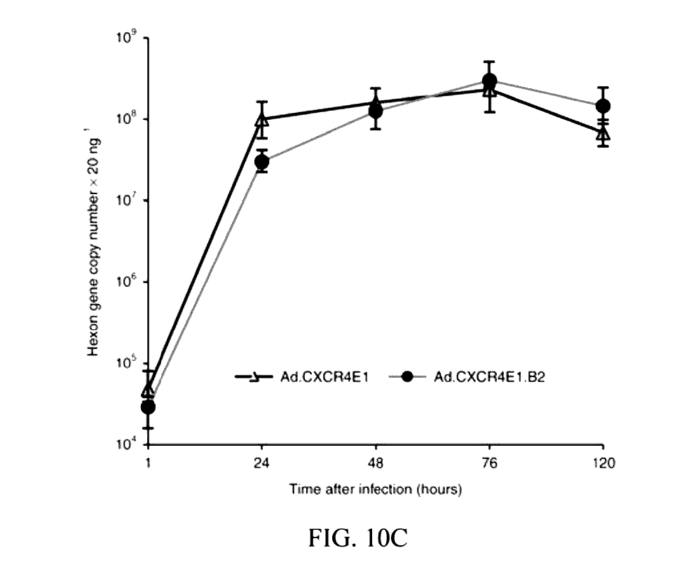

CXCR4 promoter activity was evaluated for different cell lines by infection with Ad.CMVLuc and Ad.CXCR4Luc, encoding the Luc gene under control of the CMV or CXCR4 promoter, respectively (FIG. 10A). Relative Luc expression following infection of human cancer cells with either Ad.CMVLuc or Ad.CXCR4Luc. Luciferase activity was measured in cell lysates at 48 hours after infection. Data are presented as mean±SD. RLUs, relative light units (FIG. 10A). Levels of Luc expression varied in different cell lines in proportion to viral doses of infection (results not shown). Infection with Ad.CXCR4Luc yielded lower Luc expression in comparison with Ad.CMVLUC. Additionally, ratios of Luc expression in cancer cells following Ad.CMVLuc and Ad.CXCR4Luc infection were calculated. Average ratios for all the individual sets of numbers for different cancer cells were compared. As shown in FIG. 10B, HS766T cells demonstrated high CXCR4-to-CMV ratio of Luc expression in comparison with LS174T and THLE-3 cells, whereas U118MG and U118-hCAR cells showed the lowest CXCR4-to-CMV ratios. The CXCR4-to-CMV ratios of Luc expression in human cells following infection with Ad.CMVLuc or Ad.CXCR4Luc. Data points represent the mean±SD of a representative experiment. Thus, all tested cells demonstrated levels of CXCR4 activity suitable to facilitate replication of CXCR4-driven CRAds.

FACS analysis of human colorectal adenocarcinoma LS174T cells revealed relatively high levels of hCAR and hCEA expression (Table 1). Taking into consideration the results of previous experiments, LS174T cells were selected for subsequent evaluation of Ad.CXCR4E1,B2 and Ad.CXCR4E1 replication. For this study, LS174T cells were infected with either Ad.CXCR4E1.B2 or Ad.CXCR4E1, then cells and media were collected at 1, 24, 48, 76, and 120 hours after infection. Human, colorectal adenocarcinoma LS174T cells were infected with 1×10 ³ vp per cell and harvested on indicated time points. Total DNA was isolated and hexon gene copy number was obtained using quantitative PCR. Data are presented as mean±SD. Replication was measured by evaluating the presence of the adenoviral hexon gene with qPCR. Both vectors show efficient replication, with the hexon gene copy number increasing ˜1,000-fold in the first 24 hours after infection (FIG. 10C). Thus, these data demonstrate that retargeting through incorporation of sdAb allows Ad.CXCR4E1.B2 to replicate in tumor cells. Of note, the level of replication achieved compared to Ad.CXCR4E1 with wild-type fiber.

### Example 11

This example illustrates Ad.CXCR4E1.B2 selectively induces tumor cell lysis.

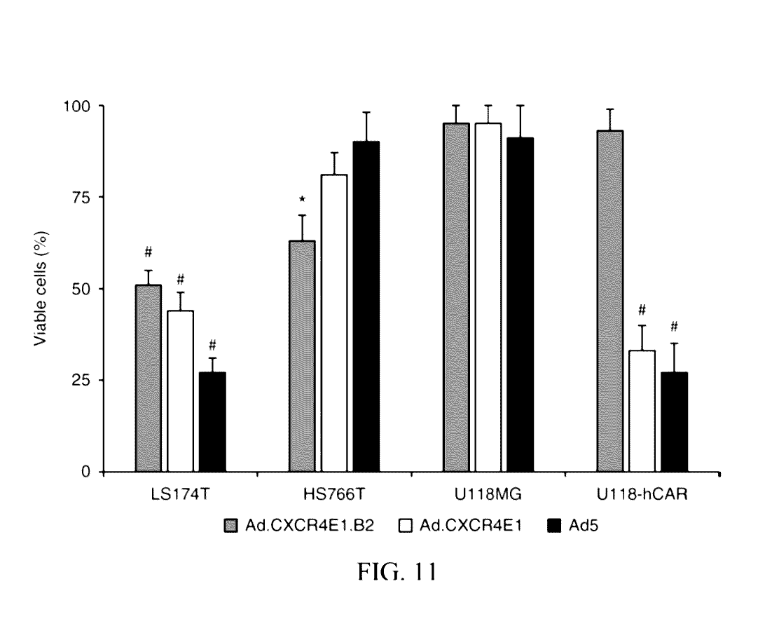

To evaluate whether specific replication in hCEA positive, CXCR4 positive tumor cells resulted in subsequent cytolysis by Ad.CXCR4E1.B2, a cytotoxicity assay was performed. To measure cytotoxicity of the sdAb-retargeted CRAd, cells were seeded into 96-well tissue culture plates at 5×10³ cells per well, incubated for 24 hours and infected with CRAd vectors at 1×10³ vp per cell. After 120 hours, cell culture medium was removed and surviving cells were fixed, and stained with 1% crystal violet (Sigma-Aldrich, St Louis, Mo.) in 70% ethanol for at least three hours at 25° C. The plates were extensively washed in tap water, air dried and optical density was measured at 595 nm using an EL 800 Universal Microplate Reader (BIO-TEK Instruments, Wmooski, Vt.). The percentage viable cells was calculated for infected cells relative to uninfected cells. Different cancer cell lines were evaluated for hCAR and hCEA surface expression using FACS analysis (Table 1). Based on these findings, colorectal adenocarcinoma LS174T, pancreatic carcinoma HS766T, glioma U118MG and U118-hCAR cells were infected with 1×10³ vp per cell of Ad.CXCR4E1.B2, Ad.CXCR4E1 or wild-type Ad5. Five days (120 hours) after infection viable cells were evaluated using a crystal violet staining assay as described supra. As shown in FIG. 11, infection with Ad.CXCR4E1.B2 resulted in increased cytotoxicity in hCEA(+) LS174T and HS766T cells in comparison with hCEA(−) U118MG and U118-hCAR cells, while the control Ad.CXCR4E1 and wild-type Ad5 viruses were able to produce cell killing in hCAR(+) LS174T and U118-hCAR cells. In contrast, no cytolysis for either of the vectors was observed in human glioma U118MG cells deficient for hCEA and hCAR expression (FIG. 11). Number of viable cells is given as percentage of the cell number of uninfected control. The hCEA and hCAR expression status of the cell lines is as follows: LS174T: hCEA(+)/hCAR(+); HS766T: hCEA(+)/hCAR(−); U118MG: hCEA(−)/hCAR(−); U118-hCAR: hCEA(−)/hCAR(+). Data are presented as mean±SD (*P<0.05 versus U118MG cells; #P<0.01 versus U118MG cells). Of interest, Ad.CXCR.4E1 infection resulted in a modest increase of HS766T cell killing in comparison with U118MG cells (both cell lines demonstrate a low levels of hCAR expression), probably due to different levels of CXCR4promoter activity in these cells, CXCR4-to-CMV ratio of Luc expression in HS766T and U118MG cells was 0.14±0.009 and 0.02±0.011, respectively (FIG. 10B). Taken together, these findings indicate that infection with Ad.CXCR4E1.B2 induces efficient cytolysis uniquely in hCEA expressing tumor cells.

### Example 12

This example illustrates that Ad.CXCR4E1.B2 adds an additional level of specificity to limit off-target cytotoxicity in normal cells in vitro.

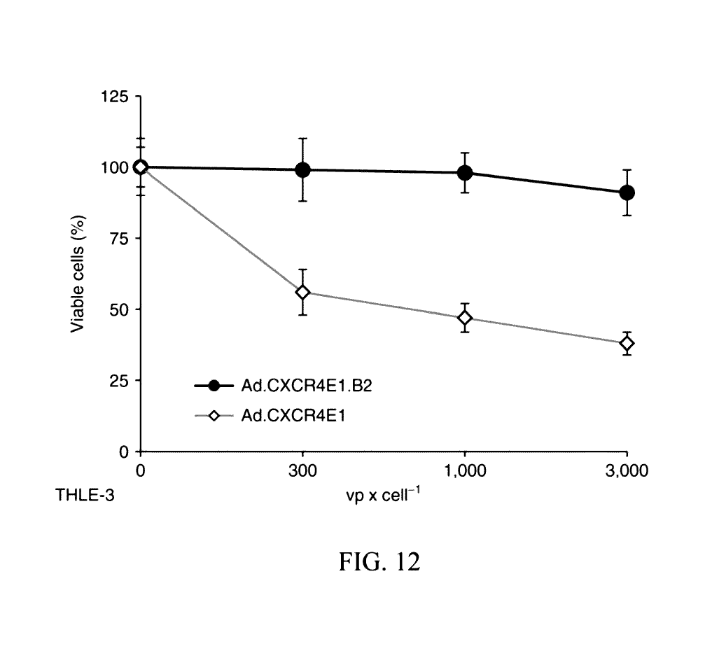

For this analysis, we evaluated the hCAR and hCEA surface expression of normal immortalized liver THLE-3 cells using FACS. As shown in Table 1, THLE-3 cells resembled a “normal cell phenotype”: hCAR positive and hCEA negative. To demonstrate the additional level of specificity of sdAb-targeted CRAds compared to wild-type fiber containing CRAds, THLE-3 cells were infected with increasing concentrations of either Ad.CXCR4E1.B2 or Ad.CXCR4E1, Cytotoxicity was determined five days (120 hours) after infection, using a crystal violet staining (as discussed in Example 11). As shown in FIG. 12, in contrast to the CAR-dependent Ad.CXCR4E1 vector, Ad.CXCR4E1.B2 demonstrated low levels of cytotoxicity at all indicated concentrations. Number of viable cells is given as percentage of the cell number of uninfected samples. These data indicate that the sdAb-mediated transductional retargeting adds an additional level of specificity to CRAds, thereby limiting off-target cytotoxicity.

### Example 13

This example illustrates that cytotoxicity by Ad.CXCR4E1.B2 is hCAR-independent and inhibited by soluble hCEA.

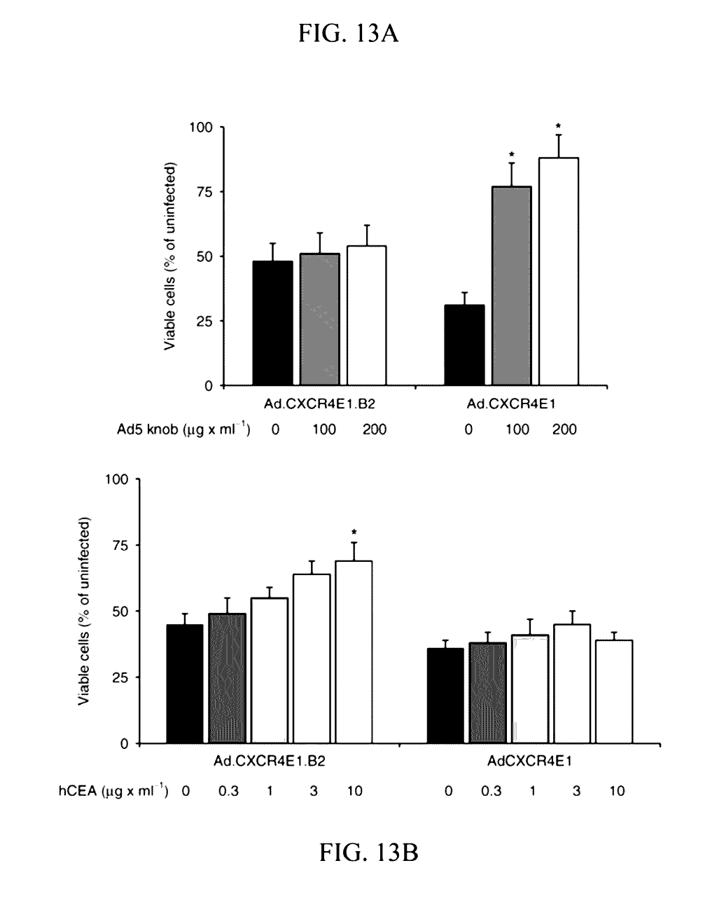

To demonstrate that Ad.CXCR4E1.B2 infection is hCAR-independent and hCEA-dependent, competition experiments were performed. To block hCAR specific transduction, cells were seeded 1×10⁵ cells per well in a 24-well tissue culture plate and incubated after one day with 100 or 200 μg/ml of soluble Ad5 knob protein for 1 hour at 4° C. before infection with Ad.CXCR4E1.B2 or Ad.CXCR4E1 at 2×10³ vp per cell After 120 hours, the cells were stained with crystal violet as described above. To block hCEA specific transduction, cells were seeded 1×10⁵ cells per well in a 24-well tissue culture plate. Both Ad.CXCR4E1.B2 and Ad.CXCR4E1 were incubated with 0.3, 1, 3, or 10 μg/ml of recombinant hCEA protein (ab742, Abeam, Cambridge, Mass.) for 30 minutes at room temperature. Afterwards cells were infected with the virus-hCEA mixture at 2×10³ vp per cell After 120 hours, the cells were stained with crystal violet as described above. Preincubation of tumor cells with soluble Ad5 knob protein was not able to block tumor cell cytolysis in Ad.CXCR4E1.B2 infected cells. However, cytotoxicity of control Ad.CXCR4E1 vector was efficiently blocked by incubation with the Ad5 knob protein (FIG. 13A). Human colorectal adenocarcinoma ES174T cells were preincubated with soluble Ad5 knob protein at indicated concentrations and infected with 2×10³ vp per cell of Ad.CXCR4E1.B2 or Ad.CXCR4E1.. Cytotoxicity was determined at 120 hours alter infection using a crystal violet staining assay. Number of viable cells is given as percentage of the cell number of uninfected samples. Data are presented as mean±SD (*P<0.01 versus no treatment). Preincubation of the vectors with hCEA protein was able to efficiently block tumor cell death for Ad.CXCR4E1.B2 but not for control Ad.CXCR4E1 vector FIG. 13). Ad.CXCR4E1.B2 and Ad.CXCR4E1 were incubated with hCEA at indicated concentrations. LS174T cells were infected with 2×10³ vp per cell of Ad.CXCR4E1.B2 or Ad.CXCR4E1. Collectively, these data show that Ad.CXCR4E1.B2 has enhanced tumor specificity for hCEA positive tumor cell lines compared to endogenous targeted CRAds and is able to cause subsequent oncolysis.

### Example 14

This example illustrates that a vector of the present teachings can be used to target dendritic cells.

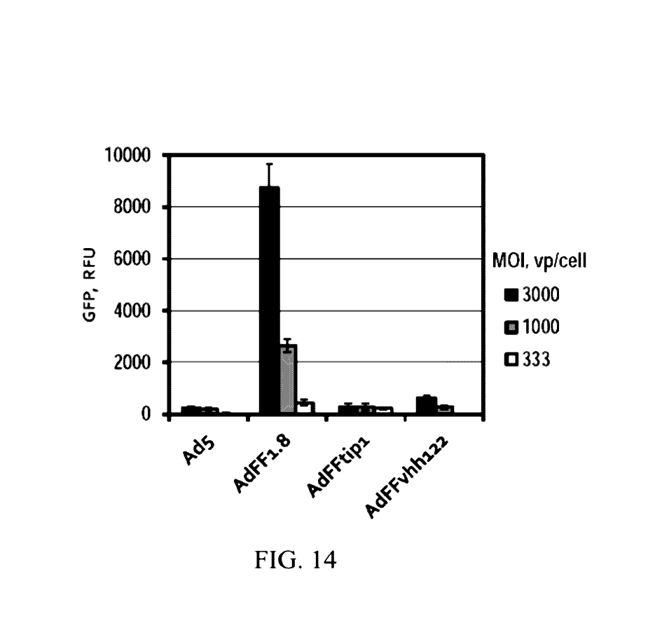

In these experiments, the present inventors provided an adenovirus vector comprising sequences encoding GFP and a camelid antibody against Nb-DC1.8 (described in De Groeve, K., et al., J. Nuclear Medicine, 51, 782-789, 2010) incorporated into the Ad5FF1.8 capsid (Ad5GFP-FF1.8), DC 1.8 can have a sequence QVQLQESGGGLVQPGGSLRLSCAASGFTFSNYGLRWVRQAPGKGLEWVAGVNGRG DVTSYADSVKGRFTISRDNAKNTLYLQMNGLKPEDTAVYYCSFIEIDGSLRKGQGTQ VTVSS (SEQ ID NO:31), Expression of Nb-DC1.8 was validated via Western blot analysis. Cells of murine dendritic cell (DC) line DC2.4 were infected with Ad5, AD5FF-TIP1 controls or Ad5GFP-FF1.8. Infection was monitored via fluorescent assays. Ad5FF1.8 showed statistically significantly increased transduction of the GFP relative to controls (FIG. 14).

### Example 15

This example illustrates that a vector of the present teachings can he used to target bone marrow dendritic cells (BMDCs).

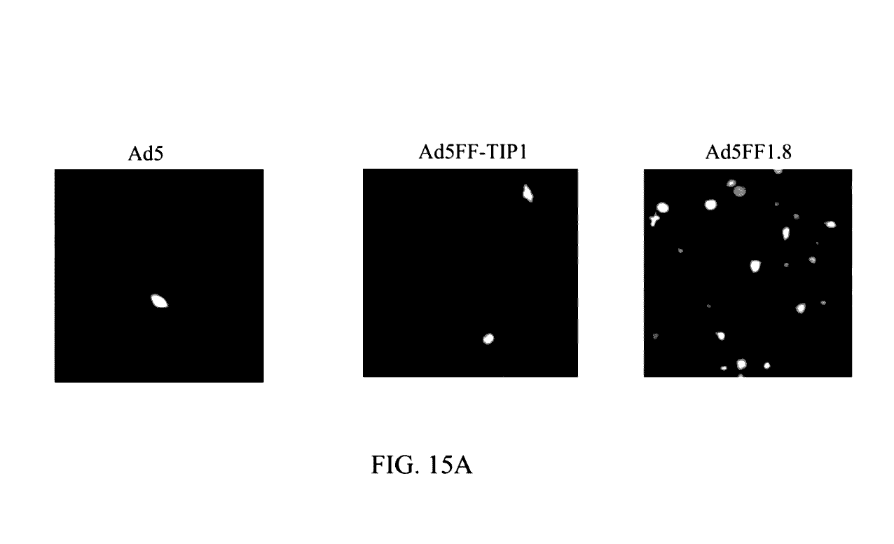

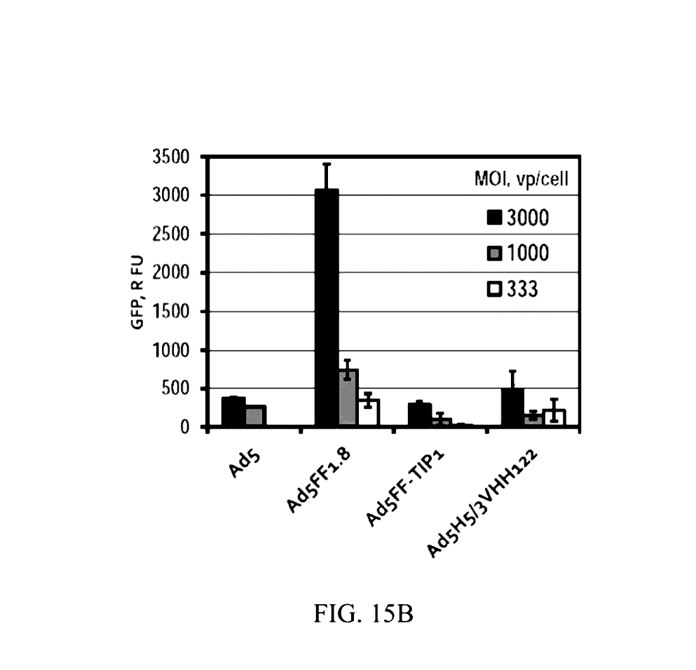

In these experiments, adenovirus vectors described supra and Ad5H5/3VHH122 were used to infect BMDCs. Expression was monitored using fluorescence microscopy (FIG. 15A). The dendritic cell targeted Ad5FF1.8 showed statistically significantly higher transduction of GFP gene compared to adenovirus lines that were not targeted to dendritic cells (FIG. 15B). The data of Examples 14 and 15 indicate that dendritic cells can be targeted by vectors of the present teachings and that a vector harboring a camelid antibody can effect transduction and expression in dendritic cells.

### Example 16

This example illustrates Ad5GFP-FF1.8 can induce interferon production in dendritic cells.

In these experiments, the inventors infected C57BL/6J female mice (n=5) with Ad5GFP-FF1.8, Ad5GFP/luc (no fibritin or DC targeting sequence), Ad5GFP-FF (fibritin without ligand) and a PBS negative control. Mice were then immunized against GFP, and the spleens of the infected mice were harvested. Immunogenicity was measured via FACS analysis and immunodetection of INFγ levels. A statistically significant increase in INFγ was observed. These experiments illustrate that a wide variety of camelid antibodies can be used in the present teachings.

All publications cited in this application are herein incorporated by reference in their entirety as if each individual publication, patent, patent application or other reference were specifically and individually indicated to be incorporated by reference.

What is claimed is:

1. A polypeptide comprising, in N-terminal-to-C-terminal order;
an N-terminal segment of Ad5 fiber tail sequence;
at least 2 pseudorepeats of an Ad5 fiber shaft domain sequence;
a portion of a third Ad5 fiber shaft domain sequence;
a carboxy-terminal segment of a T4 fibritin bacteriophage trimerization domain sequence;
a linker sequence; and
a camelid single chain antibody sequence.

2. A polypeptide in accordance with claim 1, wherein the carboxy-terminal segment of the T4 fibritin bacteriophage trimerization domain sequence comprises an α-helicaJ domain and a foldon domain.

3. A polypeptide in accordance with claim 1, wherein the N-terminal segment of Ad5 fiber tail sequence is a sequence having at least 70% sequence identity with SEQ ID NO.1.

4. A polypeptide in accordance with claim 1, wherein the at least 2 pseudorepeats of an Ad5 fiber shaft domain sequence is a sequence having at least 70% sequence identity with SEQ ID NO:2.

5. A polypeptide in accordance with claim 1, wherein the portion of a third Ad5 fiber shaft domain sequence is a sequence having at least 70% sequence identity with SEQ ID NO:3.

6. A polypeptide in accordance with claim 1, wherein the carboxy-terminal segment of a T4 fibritin bacteriophage trimerization domain sequence is a sequence having at least 70% sequence identity with SEQ ID NO:4.

7. A polypeptide in accordance with claim 1, wherein the linker sequence comprises the sequence (GlyₙSer)ₘ, n Is an integer from 2 to 6, and m is an integer from 1 to 5.

8. A polypeptide in accordance with claim 1, wherein the camelid single chain antibody sequence is against a human carcinoembryonic antigen.

9. A polypeptide in accordance with claim 1, wherein the camelid single chain antibody sequence is selected from the group consisting of a sequence having at least 70% sequence identity with SEQ ID NO:6, a sequence having at least 70% sequence identity with SEQ ID NO:7, a sequence having at least 70% sequence identity with SEQ ID NO:8, a sequence having at least 70% sequence identity with SEQ ID NO:9, a sequence having at least 70% sequence identity with SEQ ID NO.10, a sequence having at least 70% sequence identity.

10. A polypeptide in accordance with claim 1, wherein the camelid single chain antibody is a sequence having at least 70% sequence identity with SEQ ID NO:11.

11. A polypeptide in accordance with claim 1, wherein the camelid single chain antibody is DC1.8 (SEQ ID NO:31).

12. A polypeptide in accordance with claim 1, wherein the at least 2 pseudorepeats of an Ad5 fiber shaft domain sequence comprises a third pseudo-repeat of the Ad5 fiber shaft domain, wherein the third pseudo-repeat is joined to the carboxy-terminal portion of a T4 fibritin protein sequence at a fragment of an insertion loop preceding a fifth coiled-coil segment of an α-helical central domain of the T4 fibritin bacteriophage trimerization domain.

13. A nucleic acid encoding the polypeptide of claim 1.

14. An adenovirus vector comprising the polypeptide of claim 1.

15. An adenovirus vector in accordance with claim 14, wherein the adenovirus further comprises a therapeutic gene.

16. A method of treating a neoplastic disease in a subject, comprising: administering a therapeutically effective amount of a vector comprising a polypeptide in accordance with claim 1.

17. A method in accordance with claim 16, wherein the camelid single chain antibody sequence is against a human carcinoembryonic antigen.

18. A method of delivering a therapeutic adenovirus to a tumor cell, comprising: administering to a subject a therapeutically effective amount of a vector comprising a polypeptide in accordance with claim 1.

19. A method of delivering a therapeutic adenovirus to a tumor cell in accordance with claim 18, wherein the camelid single chain antibody sequence is against a human carcinoembryonic antigen.

20. A method in accordance with claim 18, wherein the therapeutically effective amount of the vector is an amount effective for killing the tumor cell.


SEQUENCE LISTING (31 sequences)

SEQ ID NO: 1 - protein, 44 aa, Adenovirus
MKRARPSEDTFNPVYPYDTETGPPTVPFLTPPFVSPNGFQESPP

SEQ ID NO: 2 - protein, 30 aa, Adenovirus
GVLSLRLSEPLVTSNGMALKMGNGLSLDEA

SEQ ID NO: 3 - protein, 8 aa, Adenovirus
GNLTSQNV

SEQ ID NO: 4 - protein, 30 aa, Bacteriophage T4
GYIPEAPRDGQAYVRKDGEWVLLSTFLSPA

SEQ ID NO: 5 - protein, 5 aa, Artificial Sequence
  linker
GGGGS

SEQ ID NO: 6 - protein, 128 aa, Lama pacos
QVQLVETGGGLVQPGGSLRLSCAASGRISDINAMGWYRQAPGKQRELVAAITSVGSNYVDSVKGRFTISKDNAKNTVYLQMYSLNPEDTAVYYCNTQCGTWLVCDGRDQWGKGTLVTVSSEPKTPKPQ

SEQ ID NO: 7 - protein, 123 aa, Lama pacos
QVQLVETGGGLVQPGGSLRLSCAASESIFSTYAMGWYRQAPGKQRELVAAITTNDIANYADSVKGRFTISRDNAKNTVYLQMNSLNPEDTAVYYCNAIFPPYNYWGQGTQVTVSSEPKTPKPQ

SEQ ID NO: 8 - protein, 130 aa, Lama pacos
QVQLVETGGGLVQPGGSLRPSCTASGSIFSIYAMGWYRQASGKQRELVALITRDEVFNYADSVKGRFTISRDNAKDTVYLQMNSLKPEDTAVYYCWVETVNDHYNSGVEDYWGQGTQVTVSSEPKTPKPQ

SEQ ID NO: 9 - protein, 129 aa, Lama pacos
EVQLVESGGGFVQAGESLTLSCTSSTLSCTSSTLTFTPYRMAWYRQAPGKQRDLVADISSGDGRTTNYADFAKGRFTISRDNIKNTVFLRMTNLKPEDTAVYYCNTFVSFVGIARSWGQGTQVTVSSEP

SEQ ID NO: 10 - protein, 122 aa, Lama pacos
QVQLVESGGLVQAGGSLRPSCAASGSIFLQNAMGWYRQVPGKQRELVAAITSVDSTNYADSVKGRFTISRDNAKNTVYLQMNSLKPEDTAVYYCNAPWNSDYHWGKGTLVTVSSAHHSEDPS

SEQ ID NO: 11 - protein, 124 aa, Lama pacos
EVQLQESGGGLVQAGDSLRLSCLVSGRSFNSYTMGWFRQAPGKEREFVAAILWSGPTTYYADSVKGRFTISRDNAKNTVYLQMNSLKPEDTAVYYCAAALGVLVLAPGNVYSYWGQGTQVTVSS

SEQ ID NO: 12 - DNA, 24 bp, Lama pacos
ttaggatcccaggtgcagctcgtg

SEQ ID NO: 13 - DNA, 27 bp, Lama pacos
gggatttaaataattgtggttttggtg

SEQ ID NO: 14 - DNA, 25 bp, Lama pacos
aaaggatccgaagtccaactggttg

SEQ ID NO: 15 - DNA, 25 bp, Lama pacos
tttatttaaatcaggccgccgacga

SEQ ID NO: 16 - DNA, 22 bp, Lama pacos
agaggatccgaggtgcaactgc

SEQ ID NO: 17 - DNA, 26 bp, Lama pacos
cccatttaaatcatgaggagacggtg

SEQ ID NO: 18 - DNA, 21 bp, Homo sapiens
ccaccactgccaagctcacta

SEQ ID NO: 19 - DNA, 24 bp, Homo sapiens
ctggggtagcttgttgagttccta

SEQ ID NO: 20 - DNA, 18 bp, Adenovirus
tacgcacgacgtgaccac

SEQ ID NO: 21 - DNA, 19 bp, Adenovirus
atcctcacggtccacaggg

SEQ ID NO: 22 - DNA, 21 bp, Adenovirus
accggtcccagcgtttgacgc

SEQ ID NO: 23 - DNA, 20 bp, Homo sapiens
gaggcatcctcaccctgaag

SEQ ID NO: 24 - DNA, 20 bp, Homo sapiens
tccatgtcgtcccagttggt

SEQ ID NO: 25 - DNA, 20 bp, Homo sapiens
ccccatcgagcacggcatcg

SEQ ID NO: 26 - DNA, 21 bp, Mus musculus
agctggaggacttccgagact

SEQ ID NO: 27 - DNA, 20 bp, Mus musculus
tggcacttctcctgcacctt

SEQ ID NO: 28 - DNA, 21 bp, Mus musculus
tagacgcctgcacaagccgcc

SEQ ID NO: 29 - DNA, 17 bp, Homo sapiens
tccatctcgcagttggt

SEQ ID NO: 30 - DNA, 20 bp, Homo sapiens
ccccatcgagcacggcatcg

SEQ ID NO: 31 - protein, 117 aa, Lama glama
QVQLQESGGGLVQPGGSLRLSCAASGFTFSNYGLRWVRQAPGKGLEWVAGVNGRGDVTSYADSVKGRFTISRDNAKNTLYLQMNGLKPEDTAVYYCSFIEIDGSLRKGQGTQVTVSS